(19) United States
(12) Patent Application Publication
(10) Pub. No.: US 2007/0233026 A1
(43) Pub. Date: Oct. 4, 2007

(54) Absorbent articles with feedback signal upon urination

(75) Inventors: Donald Carroll Roe, West Chester, OH (US); Jennifer Joan Nandrea, Cincinnati, OH (US); Masaharu Nishikawa, Montgomery, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/724,838

(22) Filed: Mar. 16, 2007

Related U.S. Application Data
(60) Provisional application No. 60/788,415, filed on Mar. 31, 2006.

(51) Int. Cl. A61F 13/15

(52) U.S. Cl. 604/361; 604/364

(57) ABSTRACT

An absorbent article includes an outer cover having a longitudinal axis, a topsheet attached to the outer cover and having a body-facing surface, and an absorbent core disposed between the outer cover and the topsheet. The article further includes a sensation member that includes a sensate, which can be associated with the topsheet or may be separate from the topsheet. The sensate provides a sensation of a temperature change greater than an actual temperature change to the wearer's skin in response to a urination event. The sensate can be incorporated in combination with one or more additional sensation members, such as hydrophilic members, hydrophobic members, and tactile members. Visible and/or tactile indicia may be associated with the sensation member.

### CROSS-REFERENCE TO RELATED APPLICATIONS

This application claims the benefit of U.S. Provisional Application No. 60/788,415 filed on Mar. 31, 2006, the substance of which is incorporated herein by reference.

### FIELD OF THE INVENTION

The present disclosure generally relates to absorbent articles, including diapers, training pants, pull-on diapers, inserts, diaper holders and liners, and the like, and in particular to an absorbent article with a feedback sensation member, which may be adapted for use in urinary toilet training.

### BACKGROUND OF THE INVENTION

Absorbent articles are well known in the art. These articles typically have an absorbent assembly held or positioned in proximity to the body of a wearer during use in order to capture and absorb bodily exudates discharged from the wearer. Typical absorbent articles include a topsheet facing the wearer, which permits fluid exudates to pass through, and a backsheet, which prevents the exudates from escaping from the absorbent article.

The toilet training stage may be referred to as the “point of exit” from the diaper product category because toddlers who have successfully completed toilet training typically no longer wear diapers. The age at which children are toilet trained in “developed” countries has increased steadily over the past several decades and is now in the range of about 24-48 months. One reason for which toilet training has become delayed is that significant technical improvements have been made in diaper dryness and comfort. For example, when wearing a typical modern diaper, the child may have dry skin even after one or more occurrences of urination. As a result, the child may feel little or no discomfort and often may not even be aware that he or she has urinated.

Some parents may have the child wear cotton training pants or cotton underwear during urinary training so the child feels discomfort following urination in his or her “pants.” It is believed that such discomfort assists with learning or provides motivation to learn to voluntarily retain urine (at least until the child can urinate in a socially acceptable time/location). Cloth training pants leave the skin wet and, due to their high breathability, promote evaporative cooling of the skin, further enhancing discomfort. The current tradeoff in this approach, however, is that cloth training pants have poor urine containment, often leading to wet clothing and wet surroundings, e.g., carpeting, furniture, etc. There is a need to provide disposable absorbent articles where the article itself has features which facilitate toilet training without compromising the convenience of the disposable product form.

Several attempts have been made in the prior art to provide disposable absorbent articles which provide some sort of feedback such as a “coolness” or “wetness” signal to alert the wearer of the incidence of urination. Examples of such approaches have included garments which provide a “feel wet” signal. U.S. Pat. No. 6,958,432 discloses an example of a garment with a member which attempts to provide such a feeling of wetness. U.S. Pat. No. 6,642,427 discloses a garment which contains endothermic salts and similar substances to provide a temperature change or feeling of coolness. U.S. Pat. No. 6,929,819 discloses an article which attempts to provide a mild concussive sensation, while U.S. Pat. No. 5,797,892 discloses an article which attempts to provide a dimensional change upon urination to provide a tactile feedback to the wearer upon urination.

These approaches found in the prior art have met with some commercial success and accomplish the training objective at least to some degree. It has been found during development of the present invention that several—sometimes contradictory—characteristics of feedback mechanisms are important. For example, it has been found during development of the present invention that an ideal feedback mechanism should provide a nearly immediate response upon urination. If the response time for the signal is too long, the child may not properly associate the signal with the urination event which triggered it. It has also been found during development of the present invention that the feedback mechanism should ideally also provide a temporary signal. The temporary duration of the signal helps to reinforce the association with the urination event. In other words, if the signal lasts too long or indefinitely the wearer may become undesirably accustomed to the signal. Additionally, a signal which persists may lead to undesirable impacts such as stress on the wearer's skin.

Another characteristic of well designed feedback mechanisms discovered during development of the present invention is that such mechanisms should be harmless to the wearer and the wearer's skin. For example, a feedback mechanism which provided prolonged contact of a urine saturated component with the skin could lead to skin irritation and, therefore, would not be ideal.

It has also been found during development of the present invention that ideal feedback mechanisms be unmistakable to the wearer. For example, if the signal provided to the wearer were too subtle or ambiguous the training benefit of the signal may not be effectively achieved.

Consequently, a need, therefore, exists for disposable absorbent articles and garments which provide urination feedback mechanisms which are as immediate and unmistakable to the wearer as possible. Such feedback mechanisms should also be temporary and harmless. It would desirable to provide an article that can facilitate urinary toilet training by enhancing a wearer's awareness that urination has occurred by providing such a signal to the wearer while at the same time providing the protection of an absorbent article to prevent soiling of the wearer's clothing and surroundings.

### SUMMARY OF THE INVENTION

In accordance with one aspect of the present invention, an absorbent article is provided for wearing about the lower torso of a wearer. The article includes a first waist region, a second waist region disposed opposite said first waist region, and a crotch region connecting said first waist region and said second waist region. A water-impermeable outer cover is provided, along with a water-permeable topsheet attached to the outer cover and having a body-facing surface. An absorbent core is disposed between said outer cover and said topsheet. A sensory element member is at least partially disposed in the crotch region of the absorbent article. The sensory element member includes a sensate that provides a temperature change sensation to the wearer upon a urination event. The sensory element member includes a body-contacting portion.

In accordance with another aspect of the present invention, an absorbent article includes an outer cover having a longitudinal axis, and a topsheet attached to the outer cover and having a body-facing surface and an opposite surface facing the outer cover. An absorbent core is disposed between the outer cover and the topsheet. A sensory element member includes a sensate that is spaced a distance upwardly from the absorbent core.

In accordance with still another aspect of the present invention, an insert is provided that is removably attachable to an absorbent article. The insert includes a structural layer that is liquid permeable. A sensate is associated with at least one of a body-facing surface and a garment-facing surface of the structural layer. A fastening member is also provided that is configured to removably connect to the absorbent article.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. In the accompanying drawing figures, like reference numerals identify like elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1a is a plan view of an absorbent article with a section of a topsheet removed to expose an underlying absorbent core;

FIG. 1b is a cross-sectional view of an absorbent article similar to that illustrated in FIG. 1a showing a stretchable ear configuration;

FIG. 2 is a perspective view of an exemplary absorbent article shown in its relaxed, contracted state, i.e., with the contraction induced by elastic members;

FIG. 3a is a plan view of an absorbent article having a sensation member according to an embodiment of the present disclosure;

FIG. 3b is a cross-sectional view of the article shown in FIG. 3a illustrating the layers of the sensation member;

FIG. 4a is an isometric view of the article shown in FIG. 3a illustrating a first exemplary attachment of the sensation member;

FIG. 4b is an isometric view of the article shown in FIG. 3a illustrating a second exemplary attachment of the sensation member;

FIG. 5a is a top plan view of an insert for an absorbent article constructed in accordance with one embodiment of the present invention;

FIG. 5b is a cross-sectional view of the insert illustrated in FIG. 5a taken along line 5b-5b;

FIG. 6a is a top plan view of an insert for an absorbent article constructed in accordance with another embodiment of the present invention;

FIG. 6b is a cross-sectional view of the insert illustrated in FIG. 5a taken along line 6b-6b;

FIG. 7a is a plan view of an absorbent article having a sensation member according to another embodiment of the present disclosure;

FIG. 7b is a cross-sectional view of the article shown in FIG. 7a illustrating the layers of the sensation member;

FIG. 8a is a plan view of an absorbent article having a sensation member according to a further embodiment of the present disclosure;

FIG. 8b is a cross-sectional view of the article shown in FIG. 8a illustrating the layers of the sensation member;

FIG. 9a is a plan view of an absorbent article having an impregnated sensation member according to an alternative embodiment of the present disclosure; and

FIG. 9b is a cross-sectional view of the article shown in FIG. 9a illustrating the layers of the sensation member.

FIGS. 10a-10c are cross-sectional views of another embodiment of an absorbent article.

FIG. 11 is a cross-sectional view of an embodiment of the absorbent core.

### DETAILED DESCRIPTION OF THE INVENTION

### Definitions

As used herein, the following terms have the following meanings:

The term “absorbent article” refers to a device that absorbs and contains liquid, and more specifically, refers to a device that is placed against or in proximity to the body of the wearer to absorb and contain the various exudates discharged from the body.

The term “associated with”, in relation to highlighting, refers at least to highlighting that is on an element or to highlighting that is disposed proximate to an element.

The term “associative correlation” refers to establishing a mutual or reciprocal relation between the visible highlighting and that with which it is being associatively correlated so that an association, i.e. a mental connection or bond, is formed between the two. This term is used in the context of associatively correlating the respective visible forms of the visible highlighting and an externally visible graphics in or on the absorbent article as well as in the context of associatively correlating the visible highlighting or graphics with the concept of urinary toilet training, For example, associatively correlated graphics may serve in concert to draw attention to an opportunity for urinary toilet training when an absorbent article is viewed prior to its being worn, to provide an externally visible reminder of the presence of the sensation member in the interior of the absorbent article while it is being worn, etc. Similarly, visible highlighting that provides a visual reference to a topic related to urinary toilet training, such as dryness, wetness, or protection from wetness, may serve to associatively correlate the visible highlighting to the concept of urinary toilet training and thereby facilitate an opportunity for urinary toilet training.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods may be used to attach elements together over a particular area either continuously or intermittently.

The term “caregiver” refers to a person other than the child, such as, a parent, babysitter, family member, teacher, day care worker, or other person who is able to provide sufficient assistance to the child to complete a personal hygiene task.

The term “character image” refers to a graphic containing an anthropomorphic image, and in particular an image having or suggesting human form or appearance which ascribes human motivations, characteristics or behavior to inanimate objects, animals, natural phenomena, toys, cartoon characters, or the like. The character image may be associated with popular characters in the media, advertising or well known in a particular culture. Ideally they are characters that the user, particularly if a child, cares about and wants to identify with.

The term “coloration” refers to the arrangement or degree of coloring especially when used to visibly differentiate an object or a portion of an object in order to visibly highlight it.

The term “coloring” refers to the effect produced by applying or combining colors in and/or on an object or a portion of an object.

The term “diaper” refers to an absorbent article generally worn by infants, children, and/or incontinent persons about the lower torso and having the general form of a sheet, different portions of which are fastened together to encircle the waist and the legs of the wearer.

The term “disposable” refers to absorbent articles that generally are not intended to be laundered or otherwise restored or reused as absorbent articles, i.e., they are intended to be discarded after a single use and, preferably, to be recycled, composted or otherwise disposed of in an environmentally compatible manner.

The term “disposed” is used to mean that an element(s) is formed (joined and positioned) in a particular place or position as a unitary structure with other elements or as a separate element joined to another element.

The term “graphic” refers to a product of graphic art or a graphic representation in a pictorial form. A graphic may be a symbol, shape, image, text, or other form of indicia.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower” and “top” and “bottom”, respectively.

The term “lateral” or “transverse” refers to a direction running at a 90 degree angle to the longitudinal direction and includes directions within ±45° of the lateral direction.

The term “longitudinal” refers to a direction running parallel to the maximum linear dimension of the article and includes directions within ±45° of the longitudinal direction.

The terms “pre-literate” and “incapable of reading” are used interchangeably herein to mean the inability of a child to correctly understand, comprehend and follow prompts written in a language that the child can speak without assistance of a caregiver. The ability of a child to recognize letters and/or read one or two isolated words still means that the child is “incapable of reading” since he or she is unable to understand, comprehend and follow such written prompts, without assistance. However, this definition of “incapable of reading” does not exclude the child from being able to understand, comprehend and follow visual prompts which are presented in the form of drawings, icons, symbols, gestures, cartoons and the like.

The term “refastenable” refers to the property of two elements being capable of releasable attachment, separation, and subsequent releasable reattachment without substantial permanent deformation or rupture.

The terms “releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

The term “solid coloring” refers to the unbroken, i.e., uninterrupted, coloring of an area as contrasted with the discrete line-like form of some graphics.

The term “toilet training” refers to the development of continence, which is the ability to voluntarily retain one's urine and feces. Individuals who are incontinent are unable to voluntarily retain their bodily discharges and, instead, urinate and defecate reflexively. For example, newborn babies are incontinent. Coincident with the development of continence, children typically develop the ability to voluntarily urinate and defecate, and cease reflexive elimination. This development of continence and of voluntary elimination, in place of reflexive elimination, may be accelerated and/or guided by caregivers through associative and conditioning techniques of training the child. For the purpose of the present disclosure, the term “toilet training” is used to denote training both for continence, itself, and for the voluntary elimination that is associated with continence. It is also noted that the term “toilet training” is synonymous with the term “potty training”.

The terms “training pants,” or “pant-like garments” refer to an absorbent article generally worn by infants and incontinent persons about the lower torso and having the general form of a pair of short pants that can be applied or removed from the wearer without unfastening.

The term “unitary” refers to an absorbent article that is formed of separate parts united together to form a coordinated entity so as to not require separate manipulative parts like a separate holder and liner.

The term “visible” refers to the quality of being capable of being seen by the naked eye under conditions of normal room lighting or in natural light during the daytime. Becoming “more visible” or “less visible” means changing in visibility to a noticeable extent when viewed under a generally constant or equal lighting condition.

The term “visible highlighting” refers to the visible differentiation of an object such that it noticeably stands out from its surroundings, e.g., by differing in coloration, hue, or tint, by differing in lightness, darkness, or contrast, by differing due to the presence or absence of graphical or solid color forms, or by any other variation serving to create noticeable visible differentiation.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

The term “x-y plane” refers to the generally planar structure of a sheet material defined by its length and width and lies between the sheet material's two major surfaces regardless of whether or not the sheet material is flat or curved.

The term “z-direction” refers to the direction through the thickness of a sheet material and generally orthogonal to the x-y plane.

The term “sensory element member” is analogous to “sensation member” and “feedback response member” as used herein or in co-pending applications Attorney Docket Nos. 10352P, 10353P, 10354P, 10355P and 10356P filed on Mar. 31, 2006.

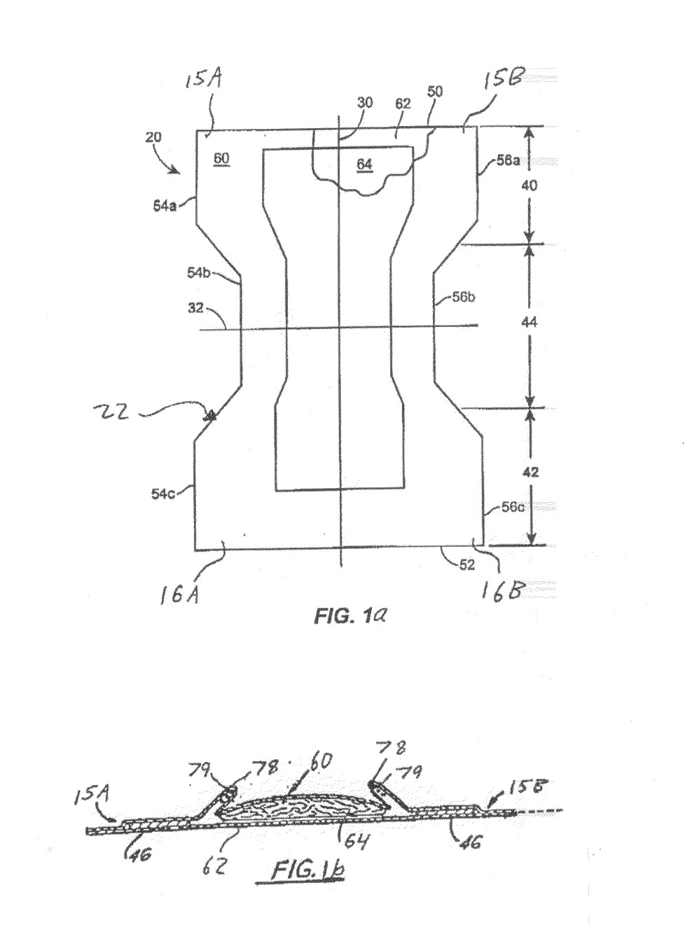

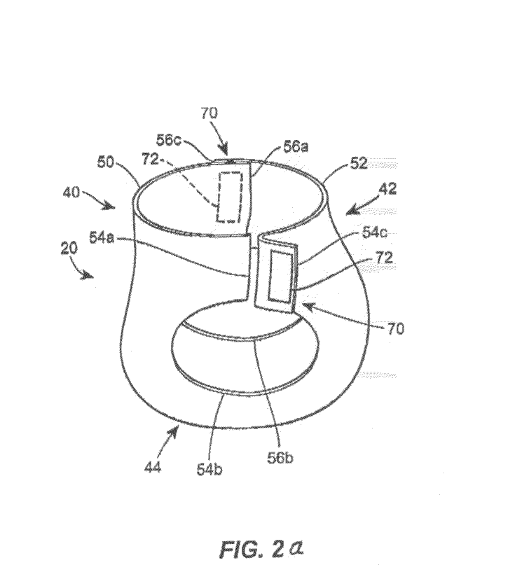

FIG. 1a is a plan view of an exemplary disposable absorbent article 20 in its flat, uncontracted state, i.e., without elastic-induced contraction. Portions of the article 20 have been cut away to more clearly show the underlying structure of the disposable absorbent article 20. As illustrated, the portion of the disposable absorbent article 20 that contacts the wearer faces the viewer (i.e., showing the interior or inner side of the article). The disposable absorbent article 20 has a longitudinal axis 30 and a transverse axis 32.

One end portion of the disposable absorbent article 20 is configured as a first waist region 40 of the disposable absorbent article 20. The opposite end portion is configured as a second waist region 42 of the disposable absorbent article 20. The waist regions 40 and 42 generally comprise those portions of the disposable absorbent article 20 which, when worn, encircle the waist of the wearer. The waist regions 40 and 42 may include elastic elements such that they gather about the waist of the wearer to provide improved fit and containment. An intermediate portion of the disposable absorbent article 20 is configured as a crotch region 44, which extends longitudinally between the first and second waist regions 40 and 42. The crotch region 44 is that portion of the disposable absorbent article 20 which, when the disposable absorbent article 20 is worn, is generally positioned between the legs of the wearer.

The disposable absorbent article 20 has a laterally extending first waist edge 50 in the first waist region 40 and a longitudinally opposing and laterally extending second waist edge 52 in the second waist region 42. The disposable absorbent article 20 has a first side edge 54 and a laterally opposing second side edge 56, both side edges extending longitudinally between the first waist edge 50 and the second waist edge 52. The portion of the first side edge 54 in the first waist region 40 is designated 54a, the portion in the crotch region 44 is designated 54b, and the portion in the second waist region 42 is designated 54c. The corresponding portions of the second side edge 56 are designated 56a, 56b, and 56c, respectively.

The disposable absorbent article 20 comprises a chassis 22 that, in turn, includes a water-permeable topsheet 60, a water-impermeable outer cover, or backsheet 62. The backsheet 62 can be breathable and, hence, water vapor permeable. The article 20 further includes an absorbent assembly or core 64, which may be disposed between the topsheet 60 and the backsheet 62 with the topsheet 60 attached to the backsheet 62. The topsheet 60 may be fully or partially elasticized or may be foreshortened so as to provide a void space between the topsheet 60 and the core 64. As explained below, a fully or partially elasticized topsheet 60 may also to tend to draw a sensation member against the skin of the wearer. Exemplary structures including elasticized or foreshortened topsheets are described in greater detail in U.S. Pat. Nos. 4,892,536; 4,990,147; 5,037,416; and 5,269,775, among others.

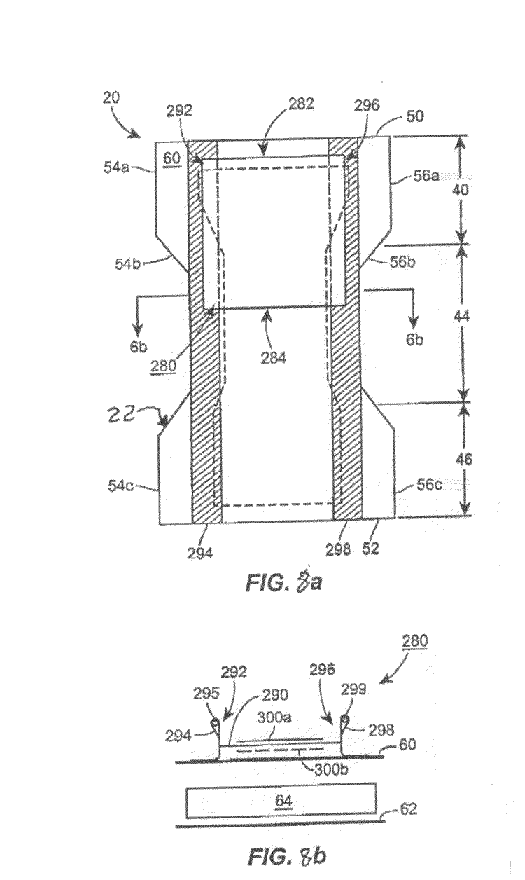

The absorbent article can further include standing leg cuffs 78, defined as an outwardly extending member including an elastic 79 that causes the member to “stands” up against the wearer's body. The standing leg cuff 78 can be permeable to liquid, impermeable, and can form a seal with respect to the wearer's body. The standing leg cuff 78 can extend longitudinally along substantially the entire length of the article 20, or can extend only a portion of the length. One example of a standing leg cuff is a barrier leg cuff as described in more detail below with reference to FIGS. 8A-B.

The absorbent article 20 further includes a first, or front, pair of ear panels 15A and 15B, and a second, or rear, pair of ear panels 16A and 16B, each ear panel extending laterally outwardly with respect to longitudinal axis 30. The ear panels 15A-B and 16A-B can be fastened to encircle the absorbent article 20 about the wearer's waist to enhance fit and reliable operation of the absorbent article 20.

The ear panels may comprise a separate element or a plurality of elements joined to the diaper 20. In one embodiment, the ear panels 62 and 64 comprise multi-directional extensible side panels, such as those disclosed in U.S. Pat. No. 5,957,908.

One elastically extensible material that has been found to be especially suitable for use in the ear panels is a laminate of at least one coverstock layer joined with an elastomeric film. (As used herein, the term “coverstock” refers to any woven or nonwoven materials.) Examples of suitable coverstock materials are hereinbefore discussed with respect to the topsheet 60 and the backsheet 62. Other suitable coverstock materials include nonwovens such as Fiberweb E004203 available from Fiberweb North America of Simpsonville, S.C., and Veratec 7pt., P-8 and P-14 available from Veratec Nonwoven Group of the International Paper Company of Walpole, Wash. Examples of suitable elastomeric films include Clopay 2870, a styrene block copolymer available from the Clopay Corporation of Cincinnati, Ohio, and Exxon 550 available from the Exxon Chemical Company of Lake Zurich, Ill. Examples of suitable synthetic foams for joining between the coverstock layers include: a) crosslinked natural rubber foams preferably having a caliper of approximately 50 mils and a density of approximately 13.3 pounds per cubic foot (0.214 grams per cubic cm), such as is available from Fulflex Inc., of Middletown, R.I., or as available from Ludlow Composites Corporation of Freemont, Ohio and marketed under the tradename Baby Foam; or b) polyurethane foams having a caliper of approximately 80 mils and a density of approximately 2.06 pounds per cubic foot (0.033 grams per cubic cm), such as is available from Bridgestone of Yokohama, Japan and marketed under the tradename Bridgestone SG Polyurethane Foam. Other suitable materials for use as or in the ear panels include structural elastic-like film (SELF) webs, as described above, natural rubber, natural rubber foams, elastomeric scrims, woven or nonwoven elastomeric webs, elastomeric composites such as elastomeric nonwoven laminates, zero strain stretch laminates, prestrained stretch laminates or the like. The above referenced U.S. Pat. No. 5,151,092 issued to Buell et al., on Sep. 29, 1992, describes suitable zero strain stretch laminates and prestrained stretch laminates.

In an alternative embodiment, the side panels may comprise a laminate including an elastomeric scrim material. In one such preferred embodiment, the ear panels may comprise a laminate of a scrim laminated between nonwovens, films, scrims, laminates or any combinations thereof. Suitable nonwovens include those mentioned above, as well as the carded polypropylene RMS 66265 available from Veratec Nonwoven Group of the International Paper Company of Walpole, Wash.; and the spunbond polyethylene available from Corovin GmbH of Germany under the trade name COROLIND. A suitable scrim is available from Conwed Plastics under the trade name REBOUND ELASTOMERIC NETTING, having between 2 and 20 strands per inch extending in the machine direction and the cross-direction. In yet another embodiment, the ear panels 62 and 64 may comprise a laminate including a formed film such as X-15301 manufactured by Tredegar Film Products, Inc. of Terre Haute, Ind.

The side panel laminate can be discretely attached to the chassis 22, and can be rendered extensible by ring rolling or activation, both processes being understood by one having ordinary skill in the art. Other stretchable ear configurations and methods of fabrication are disclosed in U.S. Pat. Nos. 6,004,306 and 5,167,897.

Alternatively, referring to FIG. 1b, the ear panels 15A-B can be formed integrally with the topsheet 60 and the backsheet 62. While ear panels 15A-B are illustrated, it should be appreciated that ear panels 16A-B are similarly constructed. A stretchable member 46 (e.g., an elastic member) is substantially incorporated with and made part of one or more of the ear panels 15A-B. The stretchable member 46 can be interposed between the topsheet 16 and backsheet 18 in the ear regions 26. The stretchable member 46 may assume a variety of shapes and sizes and may be positioned at various locations within the ear panels 1 5A-B as long as the stretchable member 46 provides the ear panels with sufficient extensibility to accomplish joinder of the ear panels 15A-B to form a waist region.

When the ear panels 15A-B are joined, there is formed an absorbent article 20 having stretchable side waist regions formed by the pairs of ear panels 15A-B and 16A-B. The stretchable member 46 is designed and is positioned within the ear panels to facilitate stretching of the ear panels in the lateral direction. The article 20, therefore, can extend around and contract toward the side waist region of the user, and effect a snug fit. In one alternative embodiment of the invention, a substantially continuous elongated stretchable member 46 is positioned along both the first waist region 40 and second waist region 42, thereby acting as a nearly continuous belt around the user.

In a unique aspect of the invention, the ear panels are attached to the stretchable member 46 when the stretchable member 46 is in a stretched or extended configuration. In forming an ear panel, the stretchable member 46 is first extended or otherwise stretched laterally and then the inside surface of the topsheet material and/or backsheet material at the ear panel is/are secured or otherwise attached to a corresponding surface of the stretchable member 46 by conventional adhesive or mechanical means. It is important to extend the sheet material(s) to an extent where it displays a substantially smooth, flat outer surface, but not necessarily to the extent where the material stretches and/or deforms. When the stretchable number 46 is then allowed to contract or relax, the topsheet material and/or backsheet material contracts with it. This ear panel configuration is described further in U.S. Pat. No. 6,702,795.

In some embodiments, the ear panel 15A can be joined to the ear panel 16A in a non-refastenable manner or in a refastenable manner such that the first waist region 40 and the second waist region 42 are joined. The ear panels 15B and 16B can be joined together in a similar fashion as that described for ear panels 15A and 16A. In some embodiments, the disposable absorbent article 20 or a plurality thereof can be sold pre-fastened in a package, whereby the ear panels can be joined non-refastenably and/or refastenably. In some embodiments, a plurality of disposable absorbent articles 20 can be sold in a package wherein some of the disposable absorbent articles are pre-fastened and some are not pre-fastened.

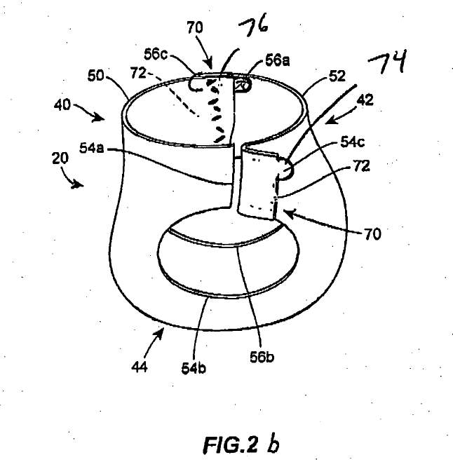

The disposable absorbent article 20 may be sealed at the sides so as to be configured as illustrated in FIG. 2a. Alternatively, the article 20 can include refastenable side seams 70 that can be used to fasten the waist regions 40, 42 together. According to one exemplary embodiment, the waist regions 40, 42 may be fastened at the sides to apply the article like a diaper. According to a further exemplary embodiment, the side seams 70 may include fasteners 72 that can be used to configure the article like a pair of pull-on training pants or disposable pants.

Any suitable fastening element known in the art can be used in the present invention. Examples of suitable fastening elements include engaging components, receiving components, adhesive components, cohesive components, the like, or any suitable combination thereof.

As illustrated in FIGS. 1 and 2a-b, the fasteners 72 may be disposed on the interior of the disposable absorbent article 20 in the second waist region 42 adjacent to the portion 54c of the first side edge 54 and adjacent to the portion 56c of the second side edge 56. The portion 54c of the side edge 54 is shown in an open condition, such as prior to closing and fastening or after being reopened. The portion 56c of the opposing side edge 56 is shown fastened, i.e., forming a pants configuration. In FIG. 2a, the second waist region 42 overlaps the first waist region 40 when they are fastened together.

The fasteners 72 may be formed of any material and in any form that will releasably attach to the mating surface of the opposing waist region when pressed against it. For example, the primary fastening component may be a mechanical fastener that releasably engages with the mating surface, such as by means of a plurality of hooks engaging with loops formed by fibers in a nonwoven sheet.

An example of a suitable engaging component may comprise hook fastening material. A hook fastening material according to the present invention may be manufactured from a wide range of materials. Examples of suitable materials include nylon, polyester, polypropylene, or any combination of these materials, or other materials as are known in the art. An exemplary hook fastening material is described in U.S. Pat. No. 4,846,815. Other suitable examples and processes for making the same are described in U.S. Pat. No. 5,540,673 and in WO 2004/082918. Alternatively, the engaging elements may have any shape such as hooks, “T's”, mushrooms, or any other shape as are well known in the art.

An example of a suitable receiving component may comprise a plurality of loops. Loop fastening material and a method for making the same are described in U.S. Pat. No. 5,380,313; U.S. Pat. No. 5,569,233; U.S. Pat. No. 5,407,439; U.S. Pat. No. 5,542,942; U.S. Pat. No. 5,669,900; U.S. Pat. No. 5,318,555; U.S. Application Publication No. 2003/0077430; and WO 04/030763.

Alternatively, the primary fastening component may be an adhesive, cohesive, or selective adhesive material that releasably adheres to the mating surface. An example of a suitable adhesive component may comprise discrete tape tabs. An example of a suitable tape tab is available from the 3M Corporation of St. Paul, Minn., U.S.A. under the designation of XMF99121. Any suitable adhesive component known in the art can be used.

An example of a suitable cohesive component may comprise cohesive fastening patches. In some embodiments, the cohesive fastening patches may be formed of an inherently crystalline water-based synthetic elastomer to which a tackifying agent has been added to disrupt the polycrystalline structure and thereby render the elastomer cohesive. Exemplary synthetic cohesive products are available from Andover Coated Products, Incorporated, of Salisbury, Mass., U.S.A. and are described in U.S. Pat. No. 6,156,424.

Still other variations are also possible. For example, the fasteners 72 may be disposed on the interior of the article 20 in the first waist region 40 such that the first waist region 40 overlaps the second waist region 42 when they are fastened together. As another example, the fasteners 70 may be disposed on the exterior of the article 20 rather than on the interior. As a further example, the fasteners 70 may be used with a specific mating fastener surface particularly suited for cooperation with the fasteners 70 (e.g., a loop layer that works with a hook fastener, or a layer particularly treated to provide a suitable contacting surface for a specific adhesive). Additionally exemplary fasteners and fastener arrangements, the fastening components forming these fasteners, and the materials that are suitable for forming fasteners are described in U.S. Published Application Nos. 2003/0060794 and 2005/0222546 and U.S. Pat. No. 6,428,526, among others. Other fastener types may include “tab and slot” type mechanical refastenable fasteners. Buttons, snaps, zippers, and other types of fasteners, including refastenable fasteners are also possible.

It has been discovered during development of the present invention that the development of dressing and undressing skills as well as the development of continence are both related to and potentially important to a successful potty training experience. For example, a child may begin to recognize the urge to urinate and have an ability to control and delay the onset of urination. If such a child has a desire to use the toilet, but is wearing a disposable absorbent article which he or she does not have the dexterity to remove readily, the child may not be able to use the toilet successfully. Therefore, it may be desirable to provide “easy open” features, such as those described further below either with or without refastenability features in a garment in combination with the sensory elements described herein to provide a garment having multiple potty training features in combination which synergistically re-enforce each other. Additionally, features which allow a child to more easily lower (or raise) the garment such as handles, printed indications of gripping features or the like such as those described in Co-pending application Ser. Nos. 11/083,606 and 11/083,607 and may also be included.

The disposable absorbent article 20 may be provided with an easy open feature such as shown in FIG. 2b. Such an easy open feature can comprise a single element or a combination of elements designed to make the seams of the article easier to open so as to remove the article. For example the easy open feature could comprise a gripping tab 74. Additionally an easy open bond line pattern 76 could be provided either alone or in combination with the gripping tab 74. The easy open bond line pattern 76 shown in FIG. 2b can act as a type of “zipper” structure allowing propagation of an opening force along the side of the article 20. In some embodiments, the easy open bond line pattern may comprise indicia which indicate where to open the article. Such indicia are described in Co-pending application Ser. No. 11/198,614 filed on Aug. 5, 2005 on behalf of Liu et al. Other easy open features could include a line of weakness, a notch or tab designed to propagate a tear, a tab gripping area or similar feature designed to allow for more easy release of a refastenable side fastener. Easy open features such as those described above can be provided if desired on articles having refastenable features or those which do not have refastenable fasteners—for example, a pant like garment with pre-formed side seams incorporating an easy open feature which can be used once, but which does not allow for refastenability once opened.

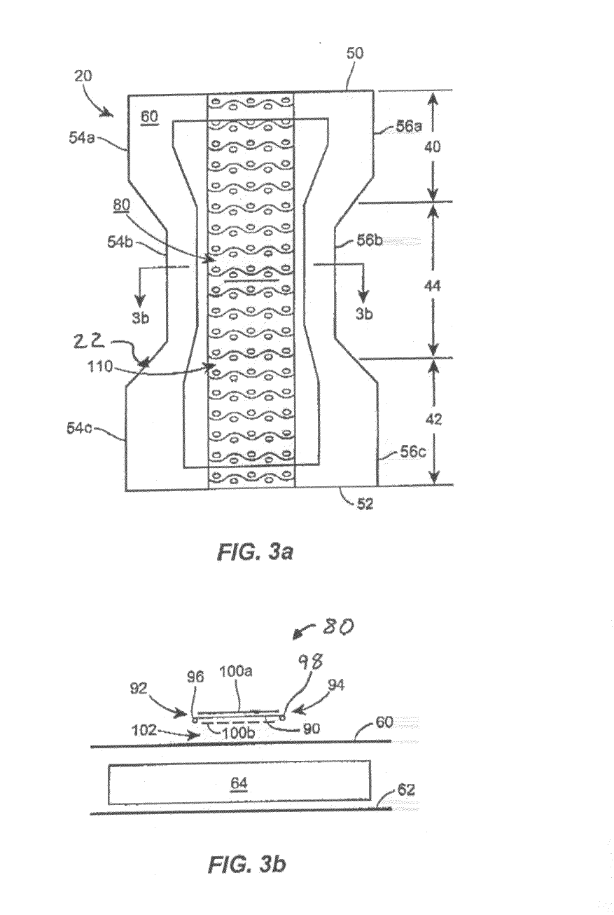

According to the present disclosure, the exemplary article 20, such as is illustrated in FIGS. 1a-c and 2a-b, may be combined with or assembled to include a sensory element member 80. Several embodiments, some with one or more variations, are illustrated in FIGS. 3a, 3b, 4a, 4b, 7a, 7b, 8a, and 8b. Elements common to all embodiments are numbered similarly in all Figures, while those elements unique to each embodiment are numbered differently, with the sensory element member according to a first embodiment being numbered as 80, a second embodiment as 180, and a third embodiment as 280. In addition, while the exemplary absorbent articles illustrated each include a single sensory element member, the articles may include a plurality of sensory element members either of the same type or of differing types according to other embodiments.

The sensory elements of the present invention may provide a feedback response to the wearer upon a urination event by the wearer. The type of feedback provided may vary, but may include by way of example a wetness sensation response or a temperature change response. A temperature change response (or “sensation”) could include one or both of an actual temperature change produced in the article which is transmitted to the wearer's skin or effected by a sensate that produces a sensation of warmness or coolness without necessarily undergoing an actual temperature change. For instance, while actual temperature changes may be effected and sensed by the wearer (i.e., changes in body heat, heat transfer from the urine, ambient environment, etc), the sensate produces a sensation of temperature change that is greater than the actual temperature change. Temperature change responses, or sensations thereof, can include increases (i.e. warmness) or decreases (i.e. coolness) from the initial or “baseline” temperature prior to the onset of the response. For purposes of clarity, a temperature response element having a response that produces an actual increase or decrease in temperature in at least a portion of the article will be referred to herein as a “temperature change” or “cooling” or “warming.” A temperature response element having a response that produces only a sensation of wetness or coolness without an actual corresponding temperature change will be referred to herein as a “sensate” which, for instance, can produce a “warmness sensation” or “coolness sensation.” Preferred sensates are those which produce a sensation of coolness.

The feedback response could include multiple sensations such as a combination of wetness and cooling. Other tactile responses such as an effervescent “bubbling” response or an audible or visual response could also be provided. Again, such tactile, audible, or visual responses may be combined with each other as desired. For purposes of clarity “tactile” responses are taken to be those corresponding to the sense of touch or feel. These include temperature change, warmness and coolness sensation, and other physical interaction with the skin responses such as bubbles, physical protrusion of the article, tightness, or the like. “Tactile” responses are distinguished from those which rely on other senses such as hearing, vision, or smell.

Tactile based sensory responses may be preferred to include in the article because a wearer may more readily associate such tactile feedback mechanisms with the triggering urination event. In particular, it may be desired to provide a temperature change response such as a cooling or a warming (or sensations of either or both) as the sensory feedback mechanism. It may be desirable to combine such wetness or temperature (or other) sensory feedback mechanisms with visual or audible response mechanisms to allow participation in the toilet training process by a caregiver or for additional re-enforcement to the wearer. Any of the mechanisms (described in greater detail below) for providing a feedback response to a wearer may be incorporated into any of the embodiments described below for structurally delivering the feedback. As noted previously, it is desirable that the urination triggered feedback responses provided by immediate, temporary, harmless, and unmistakable. Accomplishing these objectives requires control of both the manner in which the signal itself is generated, as well as the manner in which the signal is transmitted to the wearer. The experience of the wearer of the feedback signal will be based upon a combination of the feedback characteristics (such as signal intensity) and transmission characteristics.

For example, if the feedback signal provided is cooling, the experience of the wearer may be determined by a combination of the signal intensity (such as the amount of temperature change a cooling member provides) and the transmission of such coolness to the skin (such as through the pressure and body contact a sensation member incorporated into the article achieves due to its structure). Articles of the present invention balance both the signal itself and its transmission to the wearer to achieve desirable balance in signal onset, duration, noticeability, and harmlessness.

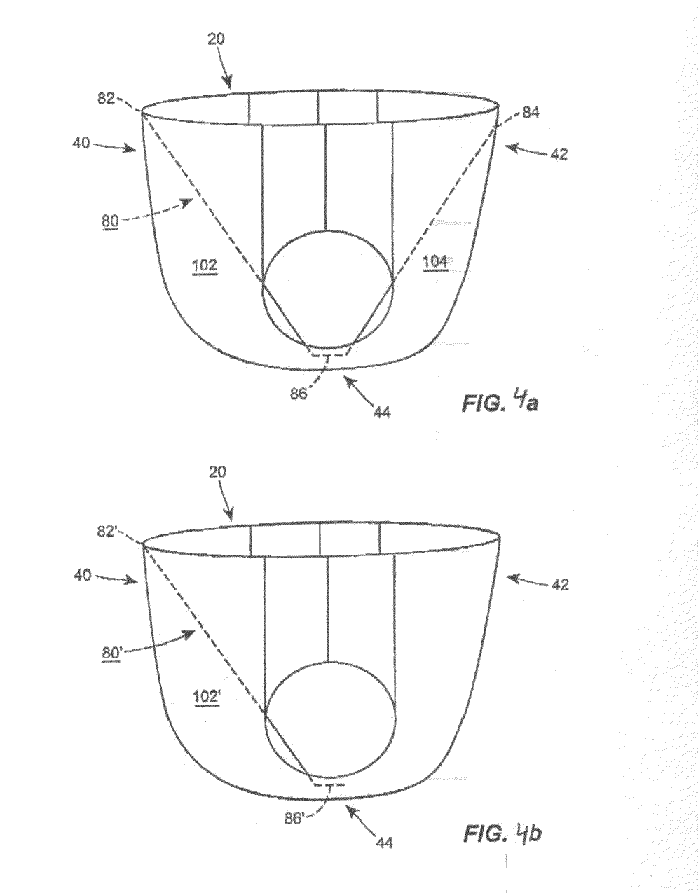

Turning then to the first embodiment of the exemplary sensory element member 80, shown in FIGS. 3a, 3b, and 4a, the sensory element member 80 illustrated is a structure that is formed separately from, but discretely attached to, the topsheet 60. In particular, and as seen best in FIG. 4a, the sensory element member 80 has a first laterally extending joining region or end 82 attached to the first waist region 40, and a second longitudinally opposing and laterally extending joining region or end 84 attached to the second waist region 42. In addition, the sensory element member 80 may have a center joining region 86 that may be attached to the crotch region 44. It is believed that the attachment of the element member 80 to the crotch region 44 may assist in stabilizing the element member 80, in facilitating fitting of the article 20 to the wearer, in preventing interference with bowel movements and in ensuring good contact of the element member 80 with the wearer's skin.

In one embodiment, the sensory element may provide a urination triggered feedback response based upon a sensation of wetness to the wearer. An example of such a wetness based feedback response is shown in FIG. 3b. As seen in FIG. 3b, the sensory element member 80 may comprise a hydrophilic layer 90, alone or in combination, as explained in greater detail below. Exemplary materials suitable for use in the layer 90 include nonwovens, foams, woven materials, etc. In particular, the layer 90 may comprise, by way of illustration and not limitation, rayon, Lyocell and other cellulose-based materials, cotton, polyester, polypropylene and polypropylene blends (e.g., with other listed materials, such as a Lyocell/polypropylene blend), and hydrophilic forms of nonwovens such as SM (spunbond meltblown), SMS (spunbond meltblown spunbond), and SMMS (spunbond meltblown meltblown spunbond). Exemplary basis weight ranges for these materials are 10-35 gsm and exemplary bulk density ranges are 0.06-0.15 g/cm³. Such materials include Nonwoven Core Cover (10 gsm SSMMS PP) manufactured by Avgol and a Lyocell/PP blend manufactured by Albis with 5-25% Lyocell/75-95% PP (ex. 25 gsm with two layers: 15 gsm philic carded layer with 12 gsm PP and 3 gsm Lyocell; 10 gsm phobic spunbond PP layer below the 15 gsm carded layer).

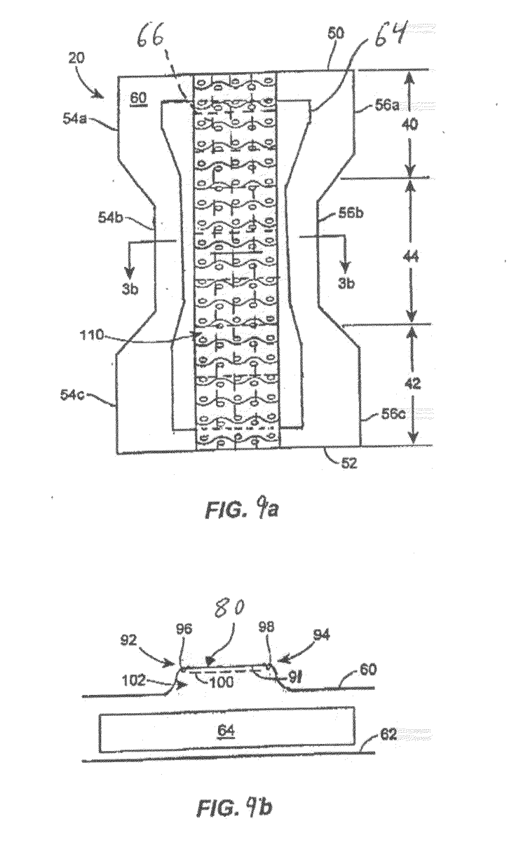

Additional examples of hydrophilic materials that can be included in layer 90, or that can be impregnated as described in more detail below with reference to FIGS. 9a-b, include lotions, creams and the like. Exemplary hydrophilic materials include surfactants, such as the NUWET silicone surfactant available from GE Silicones of Wilton, Conn.

The layer 90 may have first and second sides 92, 94 that may be parallel to the longitudinal axis 30 of the article 20. Preferably, but not necessarily, a first elastic member 96 may be attached to the layer 90 at the first side 92, while a second 98 elastic member may be attached to the layer 90 at the second side 94. The elastic members 96, 98 may extend along the entire length of the layer 90, or only a portion thereof. A fully or partially elasticized layer 90 may to tend to draw the sensation member 80 toward or against the skin of the wearer. Alternatively, the layer 90 may be formed to have a lesser length than another layer disposed relatively exteriorly, etc.

In one variation of this embodiment, the sensory element member 80 consists essentially of the hydrophilic layer 90. That is, it may be sufficient for the purpose of alerting the wearer to an insult of urine that a sufficient surface moisture quantity of urine be maintained for a period of time in the layer 90, thereby providing a wetness sensation to the wearer. The Wetness Density (as measured by the Wetness Density Test, below) may range from about 0.4 to about 1.5 grams at 60 seconds.

However, according to other variations of this embodiment, an active component, such as coating or agent, may be applied at 100a, 100b to the layer 90, which may be referred to as a support layer, as illustrated in solid and dashed line. Thus, according to a second variation, the sensation element member 80 may also comprise an active component in the form of a hydrophilic coating, which may be applied at 100a as shown in solid line in FIG. 3b. The hydrophilic coating may be disposed in a face-to-face arrangement with the support layer 90. Moreover, as illustrated, the hydrophilic coating may be disposed on the surface of the layer 90 closer to the wearer's skin (i.e., body-facing surface).

It will be also recognized that the hydrophilic coating may include a diverse range of materials, including lotions, creams and the like. Exemplary hydrophilic coatings include surfactants, such as the NUWET silicone surfactant available from GE Silicones of Wilton, Conn.

Further, according to a third variation, the sensory element member 80 may also comprise an active component in the form of a temperature response element (composition or structure), which may be applied at 100a. The temperature response element may be disposed in face-to-face arrangement with the support layer 90. Moreover, as illustrated, the temperature response element may be disposed on the surface of the layer 90 closer to the wearer's skin (i.e., body-facing surface). Further, the temperature response element may be disposed on the support layer 90 in place of the hydrophilic coating, in conjunction with the hydrophilic coating, or combined (e.g., mixed) with the hydrophilic coating. Further, the temperature response element may be impregnated directly into one or more layers comprising the sensory element member 80 structure such as support layer 90.

It will be recognized that the temperature response element may include those materials that produce a temperature change (i.e., involve an endothermic or an exothermic reaction), as well as those that produce the sensation that a temperature change has occurred without actually producing a temperature change. For example, the temperature response element may be a cooling agent. Further, the cooling agent may be the AQUACOOL dye manufactured by United Polymer Technology of Akron, Ohio. The AQUACOOL dye is a water-soluble dye that changes temperatures when brought into contact with water. An example of a cooling sensation material may be menthol or a menthol derivative, which chemicals are believed to provide the sensation of a temperature change, while not actually producing a temperature change. The COOLACT P and COOLACT 10 products manufactured by LIPO Chemicals of Paterson, N.J. are examples of menthol derivative products which may be suitable. Other examples of suitable temperature change agents and temperature sensation elements may be suitable temperature sensation agents may be found in U.S. Pat. No. 6,642,427.

Some materials that can produce a cooling temperature change by endothermic reaction can include Xylitol particles; salt hydrates such as sodium acetate, sodium carbonate, sodium sulfate, sodium thiosulfate, and sodium phosphate; anhydrous salts such as ammonium nitrate, potassium nitrate, ammonium chloride, potassium chloride, and sodium nitrate; organic compounds such as urea; and/or other similar materials. Some materials that can produce a heating temperature change by exothermic reaction can include a lightly cross-linked partially neutralized polyacrylic acid, aluminum chloride, aluminum sulfate, potassium aluminum sulfate, and/or other similar materials.

Temperature change materials can also include ortho esters or ketals. In various embodiments, the ketals can be those described in U.S. Pat. No. 5,348,750 issued to Greenberg, and U.S. Pat. No. 5,266,592 issued to Grub et al. Also, in some embodiments, the ketals can be menthone ketals, such as menthone-glycerol ketal, menthone-propylene glycol ketal, and menthone ketals resulting from reactions of menthone with alcohols containing 1-8 carbons or with polyols containing 2-8 carbons, including structural and optical isomers from such chemistries.

Exemplary suitable sensates that provide a cooling sensation are described in U.S. patent application Ser. No. 10/687,897 filed Oct. 17, 2003 and published Apr. 29, 2004 under Publication No. US20040081680A1, US patent applications 2004/0082654 A1, US 2004/0067970 A1, and US 2005/0049553 A1 and U.S. Pat. Nos. 4,296,255; 4,296,093; 4,226,988; 4,193,936; 4,178,459; 4,153,679; 4,150,052; 4,070,496; 4,070,449; 4,034,109; 4,033,994; 4,032,661; 4,020,153; 4,459,425; 6,267,974; 6,592,884; 6,328,982; 6,359,168; 6,214,788; 5,608,119; and 6,884,906.

Exemplary preferred sensates that provide a cooling sensation described in US 2004/0082654 A1 can be provided alone or in combination, and include ketals such as Frescolat® MGA (available from Symrise), cyclic carboxamides such as WS-3 (available as ISE 3000 from Qarôma, Inc.), aliphatic carboxamides such as WS-23 (available as ISE 1000 from Qarôma, Inc.), cyclohexanol derivatives such as Coolact P® (available from Takasago), and cyclohexyl derivatives such as TK10 (Takasago) and menthyl lactate, also known as Frescolat® ML (Symrise).

Still further suitable sensates that provide a cooling sensation that can be provided alone or in combination include 1-(2-hydroxyphenyl-)-4-(2-nitrophenyl-)-1,2,3,6-tetrahydropyrimidine-2-one, also known as Icilin available from Biomol International, and derivatives thereof; ethyl 3-(p-menthane-3-carboxamido) acetate, also known as WS-5 (available from Millenium Specialty Chemicals as WinSense™ Extra); N-(4-methoxyphenyl)-p-menthane-3-carboxamide, also known as WS-12; N-t-butyl-p-menthane-3-carboxamide, also known as WS-14; 1-glyceryl-p-menthane-3-carboxylate, also known as WS-30; ethylene glycol p-menthane-3-carboxylate, also known as WS-4; 3-(1-menthoxy)-2-methylpropane-1,2-diol; geraniol; eucalyptol; linaool; hydroxycitronellal; paramenthane-3,8-diol, also known as PMD-38 (Takasago International); menthyl pyrrolidone carboxlate, also known as Questice® (available from Quest International); 2-isopropyl-5-methylcyclohexyl-4-(dimethylamino)-4-oxobutanoate; (−)-Cubebol; N-(4-cyanomenthylphenyl)-p-menthanecarboxamide; (1R,3R,4S)-3-menthyl-3,6-dioxaheptanoate; (1R,2S,5R)-3-menthyl-methoxyacetate; (1R,2S,5R)-3-menthyl-3,6,9-trioxadecanoate; (1R,2S,5R)-3-menthyl-(2-hydroxyethoxy) acetate; (1R,2S,5R)-menthyl-11-hydroxy-3,6,9-trioxaundacanoate; (−)-Isopulegol; cis and trans p-menthane-3,8-diol, also known as Coolact 38D® (Takasago International); and Cooling Agent 10 from Taksago International menthol and derivatives.

The cooling sensate(s) are desirably delivered to the skin in a form capable of stimulating the cool-sensing nerves immediately (i.e., within 30 seconds, alternatively 15 seconds) subsequent to contact with urine. In one embodiment, the cooling sensate(s) are in solid form and are disposed proximal to, or on, the wearer-facing surface of the article 20. The sensate(s) can dissolve in the urine and thus contact the skin in an aqueous-based phase where they function to stimulate the cool-sensing nerves.

The cooling sensate(s) can be employed in the absorbent article 20 in several ways, either alone or in combination. For instance, a given sensate may be deposited or within the body-facing layer, for instance the topsheet 60, or the closely-underlying layer 100b of the topsheet 60, by coating, spraying, soaking, or immersing the topsheet 60 in an aqueous solution of the cooling agent and subsequently removing the aqueous carrier via drying, or by coating, spraying, soaking, or immersing an auxiliary layer of material (for instance a material that can comprise the topsheet 60) in an aqueous solution of the cooling agent, subsequently removing the aqueous carrier via drying, and placing the auxiliary layer immediately above the topsheet 60 as layer 100a or immediately below the topsheet as layer 100b.

Alternatively, the cooling sensates can be in particulate form and held on the layer of the article via adhesive, hydrogen bonding, or a skin care coating. Alternatively still, the cooling sensates, as a solid, liquid, or semi-solid, may be encapsulated within macro or microcapsules which break and release the cooling agent upon contact with urine. In this embodiment, it may be desirable for the encapsulating material to comprise a urine-sensitive material (such as a pH- or urea-sensitive material) so as to avoid release of the cooling agent prior to contact with urine.

The sensate can alternatively be retained between two adjacent layers of the article, for example between the topsheet 60 and layer 100a, or between the topsheet 60 and layer 100b, or between the topsheet 60 and the core 64 if layer 100b is not present.

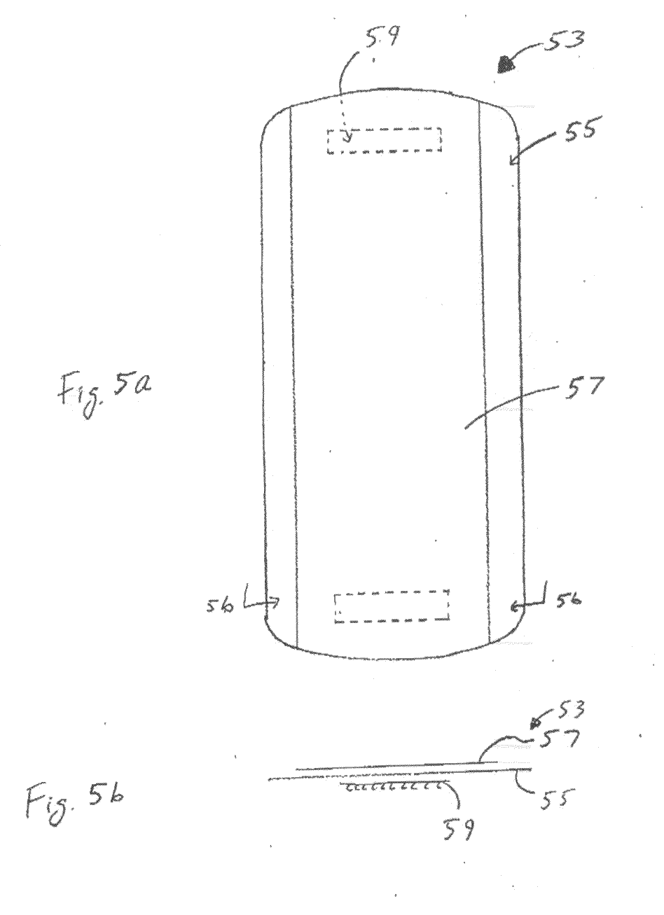

Referring to FIGS. 5a-b, the sensate can be provided via an insert 53 that can be removably attached to the article 20 as desired. For instance, the insert 53 can comprise a structural layer of material 55 (for instance any suitable material that could form the topsheet 60) that is coated and/or impregnated with the sensate 57. In the illustrated embodiment, the absorbent article 20 includes attachment zones 59 disposed on the garment-facing surface of the structural layer 55. The attachment zone 59 can comprise a fastening element such as an adhesive, cohesive, hook/loop configuration, or any alternative suitable element configured to fasten the insert 53 to the absorbent article, for instance, the body-facing surface of the topsheet 60.

Referring to FIGS. 6a-b, an insert 153 constructed in accordance with an alternative embodiment is identified with reference numerals corresponding to like elements of FIGS. 5a-b incremented by 100. The insert 153 includes the structural layer 155 in combination with the sensate 157 as described above. The insert 153 further comprises a core 64 disposed beneath the structural layer 155, and can further include a pair of laterally spaced and longitudinally extending elastic members 165, which may serve to raise the structural layer toward the wearer's body, disposed between the structural layer 155 and the core 164. Finally, the insert 153 can include a second structural layer, or backsheet member 161, disposed beneath the core 164, and an attachment zone 159 having fastening elements of the type described above for the purposes of removable attachment into an absorbent article 20.

Regardless of the mechanism used to incorporate the sensate into the article 20, at least a portion of the sensate should dissolve upon a urination by a wearer and a sufficient quantity of the dissolved sensate can contact the wearer's skin (i.e., the article desirably does not absorb or otherwise pass all of the dissolved cooling agent into the absorbent interior of the article away from the wearer).

In accordance with certain aspects of the present invention, the sensate is actively held against the wearer's skin independent of the wearer's activity or bodily position. In some embodiments, the sensate can be delivered to the wearer's skin via a transfer composition such as a skin care composition or lotion as described in U.S. Pat. Nos. 5,607,760; 5,609,587; 5,635,191; 5,643,588; 5,968,025; 6,118,041; 6,120,488; 6,120,783; 6,153,209; 6,156,024; and 6,166,285, sticky lotions as described in WO 2004/087092, adhesives such as body adhesives, and/or other semi-solid compositions that transfers to a wearer's skin via heat and/or mechanical action. In these embodiments, it may be desirable that the sensate be in a solid form so as to only contact and/or penetrate the skin in an aqueous form after contact with urine. The transfer composition can desirably be either hydrophilic enough to allow urine to contact the cooling composition or that the transfer composition only incompletely coat the particulate cooling composition.

Moreover, according to a fourth variation, the sensation element member 80 may comprise an active component in the form of a hydrophobic coating, which may be applied at 100b as shown in dashed line in FIG. 3b. According to this variation, the hydrophilic coating and/or temperature response element may or may not be included (the temperature response element being combinable with either the hydrophilic or hydrophobic agent, if present). Like the hydrophilic coating, the hydrophobic coating may be disposed in a face-to-face arrangement with the support layer 90. Moreover, as illustrated, the hydrophobic coating may be disposed on the surface of the layer 90 between the layer 90 and the topsheet 60, or the surface further from the wearer's skin (i.e., the opposite surface).

It will be also recognized that the coating 100b may include a diverse range of materials, including lotions, creams etc. Exemplary coatings may comprise hydrophobic coatings (HFC) and liquid-impermeable surface coatings (LISC). In particular, the coating may be made in accordance with the disclosure of U.S. Published Application No. 2005/0177123. Alternatively, the coatings may be acrylic polymer (e.g., acrylamide, ethyl alcohol, n-butyl alcohol, methyl-methacrylate, acrylamide, acrylonitrile, or combinations thereof) emulsions manufactured and sold, for example, under the ROHATOL tradename by Lanxess Corp. of Pittsburgh, Pa., the RH-MW1845K tradename by Rohm & Haas of Philadelphia, Pa., or the FA1, FA2, or FA3 tradenames by PolymerLatex International GmbH of Marl, Germany.

In fact, the hydrophilic and hydrophobic coatings and temperature response elements described above may be used with other sensation members, as will be discussed below. Moreover, the coatings and agents may be useful in conjunction with the structures described in U.S. Pat. No. 6,627,786, among others. As an alternative to coatings, response elements may be impregnated into any desired structure of the article to provide an integral layer having the desired response functionality.

The spacing of the first and second sides 92, 94 of the layer 90 and the width of the coating, if present, may be determined to allow enough liquid to bypass the sensory element member 80 to the core 64 so as to prevent flooding. Flooding may result in leakage of the article 20 during urination, which is undesirable in the article 20 when it is a diaper or training pant, for example. Consequently, it will be recognized that the dimensions of the layer 90 and coating may be determined to prevent flooding while at the same time wicking sufficient liquid to create a sensation of wetness for the user.

During insults of urine, the layer 90 allows urine to penetrate in the z-direction and also provides a medium for the flow of urine in the x-y plane via wicking. The layer 90 and/or the coating may enhance the movement of the passage of the urine in the x-y plane, thereby expanding the wetted area of the sensation member, which preferably is held in contact with the wearer's skin. The wicking in the x-y plane causes the urine to spread out and effectively wet a large area before being absorbed into the absorbent assembly, thereby maximizing the wetness signal experienced by the wearer.

It may be desired that the sensory element member 80 be provided with additional ingredients such as a pH buffer, enzyme inhibitors, skin care compositions including Zinc Oxide or any of the exemplary skin benefit ingredients described in U.S. Pat. Nos. 6,118,041 and 6,107,535. Such ingredients can be present to offset any impact of urine being in contact with the skin.

It is desirable that the sensory element provide a feedback response within a short time after the onset of urination. For example, the feedback response may be provided within about 60 seconds of the urination event, or may be provided within about 30 seconds or the urination event, or may be provided within about 15 seconds of the urination event. The time response to feedback onset may vary depending on the type of feedback provided. For example, if the feedback response is a wetness sensation, it may be desired that the article provide the feedback response within about 30 seconds of the urination event, or within about 15 seconds of the urination event. If the feedback response is a temperature change or temperature change sensation, it may be desirable to provide the feedback response within about 300 seconds of the urination event, or within about 15 seconds of the urination event, or within about 10 seconds of the urination event. If the feedback response is a temperature change, it may be desirable to provide a minimum rate of temperature change to produce the desired noticeability and timeliness of response. The minimum temperature change rate desired in the first 2 minutes following a urination event might be at least about 1° C./second, or at least about 2° C./second, or at least about 3° C./second, or at least about 5° C./second.

For purposes of determining the onset of a feedback response, one or more of the appropriate measurement techniques described below may be used. For example, if the feedback response provided by a particular sensory element in a particular article is a temperature change, the Temperature Change Response Measurement using a thermocouple described below may be used. If the feedback response provided is a wetness sensation, for example, the Wetness Density Test using a blotter described below may be used. These techniques provide one of skill in the art with the ability to determine when a feedback response is deemed to be “provided” (i.e. when it starts) and how long it is deemed to persist for purposes of the present application, including the claims.

If the feedback response is a temperature change, such as a temperature decrease, the temperature change should be of a sufficient magnitude to be noticed by the wearer. A temperature change of at least 5° C. (as measured from body temperate or about 37° C.) might be desired to be noticeable. Cool receptors in the skin are most sensitive at about 25° C. (representing a temperature change of about 12° C. from body temperature). Cool signal activity is still high at 20° C., and the body typically senses “cool” down to about 15° C. Temperatures lower than 10-15° C. are perceived as “cold.” It, therefore, may be desirable to provide a temperature change signal greater than about 5° C., preferably about 10-15° C., or a change of up to about 25° C. (as measured in terms of degrees change from body temperature). It may also be desired in some embodiments to have a cold signal (a large temperature change) for children who do not easily perceive more mild “cool” signals. Such a “heavy duty” or less easy to ignore cold signal could be provided by a cooling member which cools the skin to a temperature of less than about 15° C. Skin temperatures of less than 10° C., however, should be avoided since such temperatures are “noxious cold” and start to result in “burning pain.” Such signals would not accomplish one of the design objectives of “harmless” described above.

The coolness (or temperature change or temperature change sensation generally) is usually felt as long as the temperature is near the minimum temperature (in the case of coolness) resulting from the cooling agent. Once the temperature begins to increase again (or even when the rate of cooling has declined sufficiently) the cooling becomes imperceptible to the wearer. This is true even if the resulting temperature is lower than the starting temperature. As an example if the skin (or absorbent article surface) is near about 37° C. and is insulted with urine of approximately 37° C., the sensory element may trigger a temperature change response by cooling the skin to a minimum temperature of about 25° C. within about 30 seconds. As the temperature change effect begins to dissipate, and the temperature rises, the wearer may not perceive coolness once the temperature exceeds about 28° C. It will be appreciated that in this example 28° C. represents a temperature of the minimum temperature (25° C.) plus 25% of the maximum delta T (maximum temperature change initial 37° C. or 12° C. in this example). Therefore, the “duration” of the coolness based feedback may be defined in this example as the time between the urination event until the surface temperature returns to a point at which the temperature change which is no longer greater than 75% of the maximum change. The duration of this time may be about 120 seconds or less than about 150 seconds, or less than about 300 seconds. Durations under this description of about 30 seconds or about 60 seconds may be acceptable for stronger signals. The duration desired may be inversely related to the signal intensity.

In order to readily compare one article to another when such articles incorporate temperature change based feedback mechanisms, the surface temperature of the article using a thermocouple system (as described in the Temperature Change Response Measurement, below) may be recorded 30 seconds after insulting the article with 75 mL of 0.9% saline at 37° C. A typical desired temperature change at this time may range from about 5° C. to about 20° C.

If the feedback response is a wetness sensation, the amount of moisture contacting the skin should be of a sufficient quantity to be noticed by the wearer. For example, a surface Moisture Density (as measured by the Moisture Density Test) of greater than about 2 mg/cm² at 60 seconds or greater than about 4 mg/cm² at 60 seconds or greater than about 5 gm/cm²at 60 seconds may be desired. The wetness desirably does not persist for an undue period of time. For example, it may be desired that the surface Moisture Density at 10 minutes is less than about 80% or less than about 75% or less than about 70% of the surface Moisture Density measured at 60 seconds.

It is also desirable when designing the sensory element to take into account the transmission of the feedback signal to the wearer. For example, if the feedback signal is a temperature change element, the amount of temperature change element needed depends upon the degree of temperature change provided its location in the article, and how much surface area having such agent is provided in a body contacting structural member in any given body position. It has been found during development of the present invention that using the temperature change elements described above, for example, that the desired cool signal is provided when the body contacting portion of the sensory element has an area of greater than about 25 cm², or greater than about 50 cm², or greater than about 100 cm². In general, the greater the surface area of the sensory element body contacting portion, the less intense the signal itself need be to produce the desired result.

It should further be appreciated that several factors can impact the duration of the wetness signal and/or the amount of time the wetness sensation member retains liquid. For example, the capillary pressure and the absorptive capacity of the wetness sensation member can impact the duration of the signal and the amount of time the wetness sensation member retains liquid, in part. Each of these factors is influenced by several variables. Each of the factors and their variables are discussed further below.

Additionally, several factors can impact the unmistakeability of the wetness signal. In addition to the capillary pressure and the absorptive capacity of the wetness sensation member, the wicking factor of the wetness sensation member can similarly impact the unmistakeability of the wetness sensation signal.

### Capillary Pressure

The capillary pressure can impact the rate at which moisture is retained in the wetness sensation member. For example, a capillary pressure which is too high will tend to cause a retention of liquid in the wetness sensation member for longer periods of time. The higher the capillary pressure, in general, the more resistant the wetness sensation member is to giving up liquid. Therefore, a higher capillary pressure can equate to an increased duration of the wetness signal to the wearer. However, increased duration of the wetness signal can lead to prolonged exposure of the wearer's skin to urine which in turn may lead to skin health issues.

As another example, a capillary pressure which is too low can tend cause the wetness sensation member to lose liquid too quickly to provide an adequate signal to the wearer.

Wetness sensation members constructed in accordance with certain aspects of the present invention can have a capillary pressure of about 2*10⁴ N/m. In some embodiments, the capillary pressure can be about 2*10² N/m. In some embodiments, the capillary pressure can be between about 2*10² N/m to about 2*10⁴ N/m or any individual number within the range.

In accordance with one or more aspects of the present invention, the capillary pressure can be achieved through careful material selection, through treatment of the material, or through a combination of both. For example, in embodiments where the wetness sensation member comprises a nonwoven material, the pore size of the nonwoven material can impact the capillary pressure of the wetness sensation member. For example, larger pore sizes may result in a lower capillary pressure versus smaller pore sizes. The fiber chemistry can similarly impact the capillary pressure of a material. For example, polypropylene fibers may have a lower capillary pressure than pulp fibers.

In some embodiments, the capillary pressure can be achieved by treating the wetness sensation member with a surfactant, mechanically treating the wetness sensation member, and/or corona treatment of the wetness sensation member. A suitable example of mechanical treatment involves compressing the wetness sensation member. The compression of the wetness sensation member can reduce the pore size between the fibers of the material thereby increasing the capillary pressure of the material.

### Absorptive Capacity

The absorptive capacity of a material can impact the amount of liquid which the material stores. For example, for the same volume of urine, a material with a higher absorptive capacity may correlate to a higher amount of liquid which is in contact with the skin of the wearer. This in turn may lead to skin health issues. As another example, an absorptive capacity which is too low may correlate to a weak signal provided by the wetness sensation member. The absorptive capacity of the wetness sensation member can be measured in accordance with EDANA 10.4-02.

Wetness sensation members constructed in accordance with the present invention, in some embodiments, can have an absorptive capacity of greater than about 0.01 g/cm² and less than about 0.25 g/cm² or any individual number within the range. In some embodiments, the wetness sensation member can have an absorptive capacity of about 0.14 g/cm².

Similar to the discussion of capillary pressure above, the absorptive capacity of the wetness sensation member can be achieved through careful material selection, through treatment of the material, or through a combination of both. For example, in embodiments where the wetness sensation member comprises a nonwoven material, the basis weight of the nonwoven material can impact the absorptive capacity of the wetness sensation member. In general, a material having a higher basis weight has a higher absorptive capacity than the same material having a lower basis weight.

In some embodiments the absorptive capacity of the wetness sensation member can be achieved via mechanical treatment. For example, the wetness sensation member can be compressed thereby decreasing its volume. As another example, the wetness sensation member may be bonded to another substrate or the fibers of the wetness sensation member can be bonded. A material having a high bond density tends to have less absorptive capacity than does the same material having a lower bond density.

### Wicking Factor

The wicking factor may impact the amount of wetted surface area available for contacting the wearer's skin. For example, a material with a high wicking factor, e.g. 100 mm at one minute, allows for greater urine migration in an x-y plane of the material than a wetness sensation member having a wicking factor of about 10 mm at one minute. The greater the migration in the x-y plane, the more wetted surface area of the wetness sensation member is available to contact the skin of the wearer. The wicking capability of the wetness sensation member is measured in accordance with EDANA 10.4-02.

In some embodiments, the wicking factor can be greater than about 20 mm at about one minute. In some embodiments, the wicking factor can be in a range from about 20 mm at about one minute to about 100 mm at about one minute or any individual number within the range. In some embodiments, the wicking factor can be in a range from about 50 mm at about one minute to about 100 mm at about one minute. In some embodiments, the wicking factor can be in a range from between about 70 mm at about one minute to about 100 mm at about one minute.

In accordance with the present invention, the wicking factors can be achieved through careful material selection, through treatment of the material, or through a combination of both. For example, the wicking factor of a material can be impacted by the pore size between the fibers which make up the wetness sensation member. In general, the larger the pore size, the greater the wicking factor of the material.

In some embodiments, the wicking factor can be influenced through mechanical treatment. For example, the wicking of the wetness sensation member, in some embodiments, can be increased by mechanically compressing or embossing channels onto the wetness sensation member.

In some embodiments, the wicking factor of the wetness sensation member can be influenced by chemically treating the wetness sensation member. For example, a surfactant can be added to the material to both increase its wicking factor.

The signal generated by the sensation member 80 should contact the wearer's skin to elicit a sensation. In some embodiments, the absorbent article 20 and sensation member 80 are designed to enable at least intermittent, and preferably virtually continuous, contact between the wearer facing surface of the sensation member and the wearer's skin in all body positions and during all activities in which the wearer may engage. It may be desirable for the area of contact on the wearer's body to be an area having a relatively higher concentration of nerve endings. In the region of the body commonly covered by disposable absorbent articles such as pant-like diapers or training pants, the genital, perineal, perianal, inner thigh, and lower abdomen have a relatively higher nerve concentration are the preferred contact areas.

One method of promoting contact between the sensation member and the wearer is to provide a raised sensation member as described herein. In these embodiments, the skin contact is affected by providing a sensation member at least locally detached from underlying layers in at least the desired region of contact and elastically foreshortening the sensation member, or a structure to which the sensation member is affixed, causing the sensation member 80 to be lifted in the z-direction toward the body. Additionally, in certain embodiments, the elastic lifting members cause the sensation member 80 to contact the body with sufficient force and resiliency to allow the sensation member to continue to contact the body during wearer motion, or to quickly re-establish contact in the event that contact is temporarily broken.

Other methods of promoting skin contact may also be employed in place of, or in addition to the method described hereinabove. For example, at least a portion of the skin contacting surface of the article 20 may comprise a contact promoting substance that adheres gently to the wearer's skin and resists casual disengagement. Exemplary contact promoting substances may include skin care compositions, for instance lotions as described in U.S. Pat. Nos. 5,607,760; 5,609,587; 5,635,191; 5,643,588; 5,968,025; 6,118,041; 6,120,488; 6,120,783; 6,153,209; 6,156,024; and 6,166,285, sticky lotions as described in WO 2004/087092, and adhesives such as body adhesives. In certain embodiments a water-activatable adhesive may be desirable as it would only promote contact once the wearer urinates. Water activatable adhesives for use in disposable absorbent articles are disclosed in U.S. Pat. No. 6,623,465, which are incorporated by reference. The skin contact promoting substance may be disposed on at least a portion of the sensation member 80 or in a region of the topsheet 60 or other supporting structure in proximity to the sensation member 80.

Skin contact may also be promoted via resilient 3-dimensional structures comprising foams or core materials. These structures serve to hold the sensation member in contact with the wearer even during wearer motion due to their 3-dimensional resilient nature. In some embodiments, these structures may be relatively thin and unobtrusive when in a dry state and may be triggered to grow in the z-direction by contact with urine. For example, the structure may comprise a compressed foam encapsulated in water or pH sensitive material wherein the foam is allowed to expand upon contact with urine or the structure may comprise a composition capable of evolving gas held within a semi-permeable membrane such that it inflates upon contact with urine. Further examples of structures that increase in thickness upon contact with urine include those described in U.S. Pat. Nos. 3,881,491; 3,921,232; 5,330,459; 6,186,991; 5,797,892; 5,428,076; and 5,124,188.

The disposable absorbent article 20 may have indicia, such as visible highlighting 110 in FIG. 3a and illustrated as an exemplary pattern of wavy lines and circles, in the interior of the article associated with the sensory element or elements 80 to indicate the presence of the sensory element or elements 80 and thereby facilitate an opportunity for the urinary toilet training of the wearer of the article. Such visible highlighting is described in U.S. Published Application No. 2005/0096612. Although a sensory element lacking this visible highlighting is fully functional in terms of providing a noticeable wetness and/or temperature signal to the wearer, the caregiver might overlook or forget the possibility of capitalizing on each opportunity for urinary toilet training if the body-facing portion of the absorbent article presents a generally uniform appearance, such as in absorbent articles that present a generally uniform white appearance on their body-facing surfaces.

Furthermore, once the caregiver decides to mention urinary toilet training to the wearer, the visible highlighting can serve to draw the wearer's interest or can be pointed out by the caregiver and incorporated into an explanation of the upcoming opportunity. Thus, the visible highlighting can provide a topic for conversation between the caregiver and the wearer on the subject of urinary toilet training and can likewise provide a nameable object for reference by the wearer, greatly simplifying the mental task required of the wearer who desires to communicate his or her need to go to the toilet or to communicate his or her improving recognition of the wetness signal provided by the sensation member.

Even a simple solid coloring form of visible highlighting can serve to facilitate an opportunity for urinary toilet training, especially when used with wearers possessing some recognition of colors or colored forms. In addition, visible highlighting in the form of a color or colors may facilitate the teaching of recognition of colors and differences between colors and the associated learning may enhance the urinary toilet training process in turn.

In addition, the visible highlighting can serve to enhance the self-esteem of the wearer through a reminder that he or she is mature enough to be engaged in urinary toilet training. This effect can be compounded when the wearer succeeds in recognizing the need to go to the toilet and then sees the dry condition of the visibly highlighted sensation member inside the article after pulling it down.

The visible highlighting may be provided by means of printing onto a surface of the sensory element or one of its layers. For example, solid coloring or a graphic may be printed onto a surface of the coating underlying the water-permeable layer. As another example, an adhesive or a gel may be printed onto a surface of either of the two layers. Such an adhesive or gel may be colored differently from the surrounding area. Alternatively, the adhesive or gel may be uncolored or may have the same color as the surrounding area, but may still provide visible highlighting by forming a distinctive raised area or pattern and/or by surrounding a distinctive recessed area or pattern.

In addition to visible highlighting on the sensory element itself, visible highlighting or other graphical elements can be provided elsewhere on the article. This can be in addition to or instead of on the sensory element.

In some embodiments, the visible highlighting may become more or less visible when the sensation member is wetted. In addition, the visible highlighting may change color when the sensation member is wetted. Any of these effects may be created by the use of inks or dyes or other agents that undergo chemical reactions or are dispersed or concentrated when wetted by urine. In general, any of the wetness indicating compositions commonly used in externally visible wetness indicators, such as so-called “appearing” or “disappearing” wetness indicators that may become more or less visible when wetted and in wetness indicators that may change color when wetted, may be used for these versions of visible highlighting.

Wetness indicating compositions used for the visible highlighting of the sensation member may be visible from the body-facing surface of the absorbent article or may be included in the article so as to be visible from the outside, or from both. If the wetness sensation member is disposed on the interior surface of the article, a caregiver might apply different techniques to the task of urinary toilet training as compared to using an absorbent article having only a wetness indicator visible from the outside of the article. For example, while the change in an exterior wetness indicator is visible for all to see, any change in the visible highlighting of an interior sensation member remains “private” until either the caregiver or the wearer peers into the absorbent article or it is removed. Either or both approaches can be used or interchanged as desired to re-enforce the other training features described herein.

The article 20 may comprise an internal graphic 110, a first external graphic, and a second external graphic. The internal graphic may be permanent, while the external graphics may be “appearing” or “disappearing.” The first external graphics may include a character image resembling a boy and a text graphic including words forming a message, such as “Remember to go to the potty!” While the graphics may include text, the primary form of communication may be symbols, icons, or other markings other than words, so that a pre-literate child may comprehend and follow the instructions or other information indicated by the graphics, although it is not necessary for the images to be understood at this level. The second external graphics may include an image that may be associatively correlated to the permanent graphic, such as a dog or stars.

Variations regarding the internal/external graphics are possible. For example, a permanent external image may be combined with the first and second external graphics, or only one external graphic may be included. Furthermore, character images other than a boy may be provided, such as a girl, an animal (which may be anthropomorphic), a cartoon character, and the like. Still further, additional or alternative text may be provided. Additionally exemplary graphics, graphics characteristics and/or arrangements (e.g., timings, themes, scenes, storylines, etc.), the materials that are suitable for forming the graphics, and the arrangement and/or joining of these materials to the article 20 are described in co-pending and commonly assigned U.S. patent application Ser. No. 11/098,362, filed in the name of Roe et al. on Apr. 4, 2005.

Even in embodiments in which the appearance of the visible highlighting is not affected by its being wetted, the associative correlation of the respective visible forms of an externally visible marking and the visible highlighting may serve to facilitate an opportunity for urinary toilet training. For example, if both the externally visible marking and the visible highlighting have the visible form of similar graphics, the externally visible marking can serve to draw the wearer's interest or can be pointed out by the caregiver and incorporated into an explanation of the ongoing opportunity for urinary toilet training.

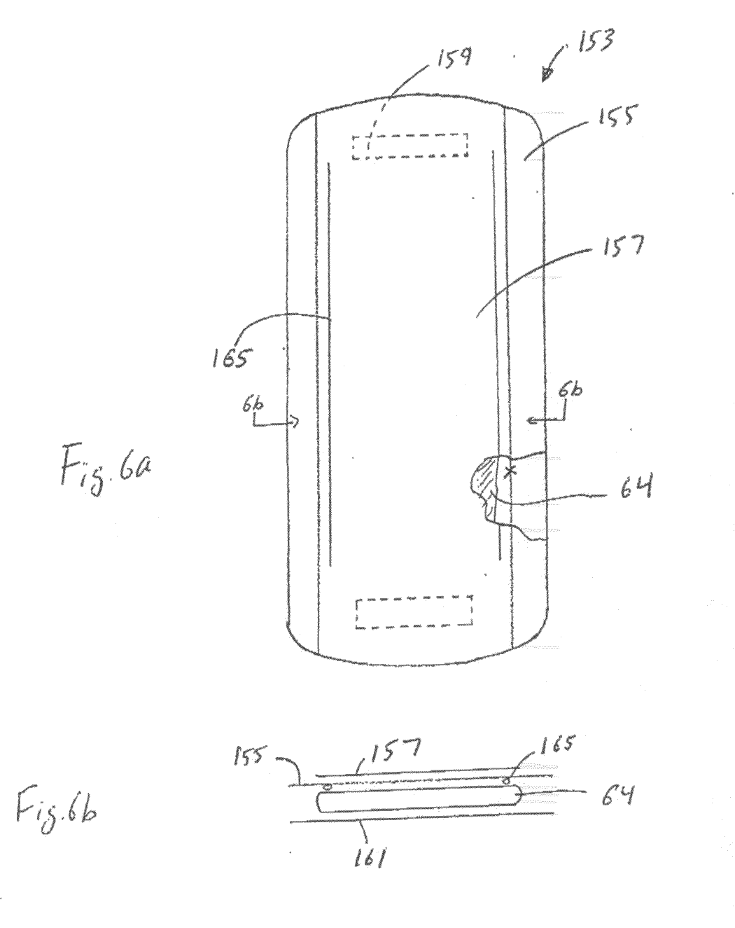

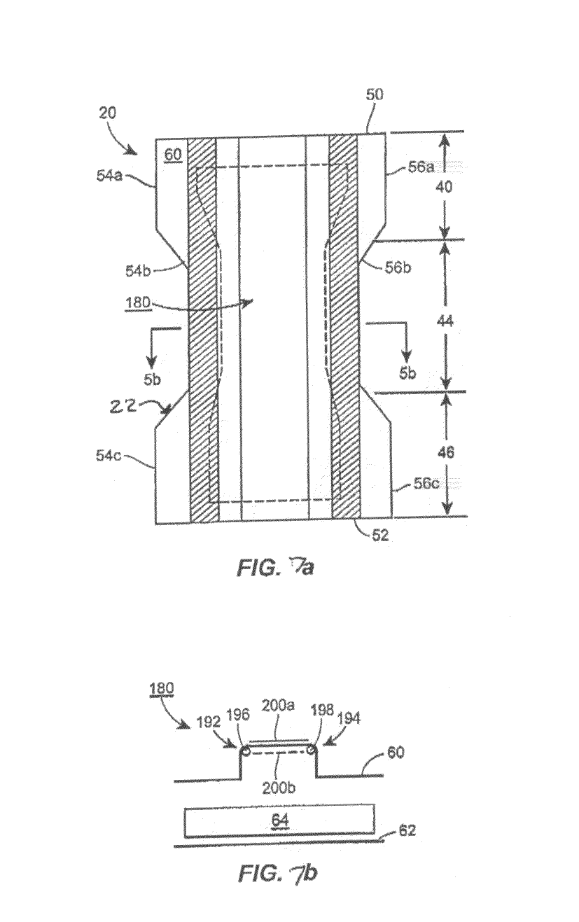

Turning next to FIGS. 7a and 7b, a second embodiment of a sensory element 180 is illustrated therein. Similar to the sensory element member 80, the sensory element 180 has first and second sides 192, 194 that are arranged parallel to the longitudinal axis 30 of the article 20. Moreover, elastic members 196, 198 may be attached to the sensory element 180 at the sides 192, 194 so as to elasticize the sensory element 180, which may assist in bringing the sensory element 180 into close contact with the skin of the wearer, increasing the efficacy and reliability of the signal transfer to the skin. Further, the sensory element may include an active component, such as (i) a coating, which may be a hydrophilic coating disposed on a body-facing surface or a hydrophobic coating disposed on an opposite surface, (ii) a temperature sensation element, which may be disposed on either surface and in substitution for, in conjunction with, or combined with the coating, and/or (iii) a layer of hydrophilic material, such as was described relative to the layer 90 above and which also may be disposed on a body-facing surface.

The sensory element 180 differs from the sensory element member 80 in that the structure corresponding to the support layer 90 is formed from a section of the topsheet 60 spaced from the core 64. That is, a section of the topsheet 60 is folded to define support layer structure of the member 180, and, in particular, is folded along the sides 192, 194. The elastic members 196, 198 are then disposed beneath the topsheet 60 in the space between the topsheet 60 and the core 64. In this fashion, the sensory element 180 may be integrated to a greater degree to the remainder of the article 20 than the member 80, thereby reducing the likelihood that the sensory element 180 will become detached from the remainder of the article 20.

The sensory element 180 may include other features in common with the sensory element member 80. For example, the coatings and agents disposed at 200a, 200b may include those exemplary coatings and agents listed above. Moreover, while not illustrated, a visible graphic 110 may be include on a surface of the sensory element 180, providing one or more of the advantages discussed above.

Turning then to FIGS. 8a and 8b, a third embodiment of the sensory element 280 is illustrated, barrier leg cuffs 294, 298 folded back slightly in FIG. 8a to expose the sensory element 280. The sensory element 280 has a first laterally extending end 282 and a second longitudinally opposing and laterally extending end 284. As will be recognized, the distance between the ends 282, 284 is shorter than the distance between the ends 50, 52, or even the distance between end 50 and the crotch region 44. According to the embodiment, the position of the ends 282, 284 relative to the ends 50, 52 and the spacing between the ends 282, 284 is such that the likelihood that the sensory element 280 will be wetted with urine is enhanced.

The sensory element 280 includes a layer 290. The layer 290 extends between the ends 282, 284. Additionally, a first longitudinal edge 292 of the layer 290 is attached to a first barrier leg cuff 294 attached to the topsheet 60, while a laterally opposed, longitudinal edge 296 is attached to a second, spaced barrier leg cuff 298, also attached to the topsheet 60. Moreover, each barrier leg cuff 294, 298 includes an elastic member 295, 299. In this fashion, it is not necessary to attach separate elastic members to the support layer 290, but the elastic members 295, 299 of the barrier leg cuffs 294, 298 instead may urge the sensory element 280 into contact with the skin of the wearer.

In fact, it is believed that the attachment of the sensory element 280 to the barrier leg cuffs 294, 298 may permit greater control over the spacing of the sensory element 280 relative to the topsheet 60 (i.e., distance between member 280 and topsheet 60) than had heretofore been possible. That is, by attaching the sensory element 280 along its sides 292, 296, rather than at its ends 282, 284, the spacing of the member 280 relative to the topsheet 60 may be better controlled than in those embodiments wherein the member is attached at its ends, or potentially even in those embodiments where the member is integrated into the topsheet 60 and elastic members disposed internal to the topsheet 60 are used to define, at least in part, the sensory element. Additionally, by attaching the sensory element 280 to the leg cuffs 294, 298, the dimension of the sensory element 280 perpendicular to the longitudinal axis may be greater than, for example, the sensory elements 80, 180 discussed above.

Similar to the embodiment shown in FIGS. 3a and 3b, the sensory element 280 may consist essentially of the layer 290, or may comprise the layer 290 in combination with a coating or an agent, which coating or agent may be disposed in a face-to-face arrangement with the layer 290, which may be referred to as a support layer. The coating may be a hydrophilic coating disposed at 300a on the body-facing surface of the layer 290, or a hydrophobic coating disposed at 300b on the opposite surface. Additionally, a temperature sensation element may be disposed at 300a on either surface and in substitution for, in conjunction with, or combined with the coating.

The sensory element 280 may include other features in common with the sensory element member 80, 180. For example, the coatings and agents may include those exemplary coatings and agents listed above. Additionally, while not illustrated, a visible graphic 110 may be include on a surface of the sensory element 280, providing one or more of the advantages discussed above.

It should further be appreciated that any of the above-described sensory elements can be constructed in accordance with an alternative embodiment. For instance, turning to FIGS. 9a and 9b, a fully or partially elasticized topsheet 60 can tend to draw a central region 91 upwards such that it is spaced from the absorbent core 64. The raised central region 91 is bound by a first side 92 and a second side 94 that both extend parallel to the longitudinal axis 30 of the article 20. A first elastic member 96 can be attached to the central raised region 91 of the topsheet 60 at the first side 92, while a second elastic member 98 can be attached to the layer 91 at the second side 94. The elastic members 96, 98 can extend along the entire length of the layer 91, or only a portion thereof.

As illustrated in FIGS. 9a and 9b, the sensory element member 80 comprises at least a portion of the topsheet 60, for instance the raised central region 91 between the elastic members 96 and 98, that is at least partially impregnated, alternatively substantially impregnated, with a material that effects a wetness sensation that is sensed at the skin of the wearer, for instance a hydrophilic material, hydrophobic material, tactile material, temperature sensation material, and combinations thereof. Accordingly, the sensory element member 80 can be drawn upwards towards or against the skin of the wearer.

It should be appreciated that the sensation element comprises one or more materials that can impregnate all or a portion of the topsheet 60. For instance, referring to FIG. 9a, the topsheet 60 can be divided into one or more impregnation zones 66 bound as indicated by hidden lines. As illustrated, the impregnation zones 66 can be laterally aligned or longitudinally aligned. Furthermore, they can be co-planar with respect to the x-y plane, or could be non-coplanar (i.e., offset in the z-direction), for instance depending upon the mode of impregnation and the material that is impregnated or coated. While each zone 66 includes a sensation material as illustrated, it should be appreciated that an individual zone or zones can be dedicated to one or more predetermined sensation materials, or not include a sensation material. Furthermore, the impregnation zones 66 can be shaped, sized, and positioned as desired to produce the desired sensation response to insults of urine.

It should be appreciated that the sensory element members 80, 180, and 280 described above could be at least partially impregnated, alternatively substantially impregnated, as described with reference to FIGS. 9a and 9b. Furthermore, any of the above-described support structures could comprise discreet layers constructed of any suitable material, for example a material that comprises the topsheet, and can be a least partially, alternatively substantially, impregnated with any sensory element as described above. Further disclosure related to impregnated sensory elements can be found in a U.S. Patent Application filed on even date herewith and entitled “Absorbent Article with Impregnated Sensation Material for Toilet Training,” Attorney Docket Number 10355P.

In addition to the features described above, the disposable absorbent article 20 may also include a variety of features known in the art, such as slit openings, outer leg cuffs, front and rear ear panels, waist cap features, elastics, and the like to provide desired fit, containment, and aesthetic characteristics. Such additional features are well known in the art and are described in U.S. Pat. Nos. 3,860,003; 5,151,092; and 6,482,191 among others. Additionally, a transfer layer, which may also be referred to as an acquisition or distribution layer, may be disposed between the topsheet 60 and the core 64. Moreover, the elements discussed above may be modified from their illustrated forms.

Various sublayers may be disposed between the topsheet and the outer cover. The sublayer may be any material or structure capable of accepting, storing or immobilizing bodily exudates. Thus, the sublayer may include a single material or a number of materials operatively associated with each other. Further, the sublayer may be integral with another element of the pull-on garment or may be one or more separate elements attached directly or indirectly with one or more elements of the disposable absorbent article. Further, the sublayer may include a structure that is separate from the absorbent core or may include or be part of at least a portion of the absorbent core.

Suitable materials for use as the sublayer may include large cell open foams, macro-porous compression resistant nonwoven highlofts, large size particulate forms of open and closed cell foams (macro and/or microporous), highloft nonwovens, polyolefin, polystyrene, polyurethane foams or particles, structures comprising a multiplicity of vertically oriented looped strands of fibers, absorbent core structures described above having punched holes or depressions, and the like. (As used herein, the term “microporous” refers to materials which are capable of transporting fluids by capillary action. The term “macroporous” refers to materials having pores too large to effect capillary transport of fluid, generally having pores greater than about 0.5 mm in diameter and, more specifically, having pores greater than about 1.0 mm in diameter.) One embodiment of a sublayer includes a mechanical fastening loop landing element, having an uncompressed thickness of about 1.5 millimeters available as XPL-7124 from the 3M Corporation of Minneapolis, Minn. Another embodiment includes a 6 denier, crimped and resin-bonded nonwoven highloft having a basis weight of 110 grams per square meter and an uncompressed thickness of 7.9 millimeters which is available from the Glit Company of Wrens, Ga. Other suitable absorbent and nonabsorbent sublayers are described in U.S. Pat. No. 6,680,422 and U.S. Pat. No. 5,941,864. Further, the sublayer, or any portion thereof, may include or be coated with a lotion or other known substances to add, enhance or change the performance or other characteristics of the element.

Embodiments of the present invention may include acquisition layers and dusting layers, each of which are well known in the art. Acquisition layer are further discussed in U.S. Pat. No. 5,460,622. Dusting layers are further discussed in U.S. Pat. No. 4,888,231. Examples of suitable configurations for leg cuffs are described in U.S. Pat. No. 3,860,003; U.S. Pat. No. 4,909,803; and U.S. Pat. No. 4,695,278.

Embodiments of the present invention may also include pockets for receiving and containing waste, spacers which provide voids for waste, barriers for limiting the movement of waste in the article, compartments or voids which accept and contain waste materials deposited in the pull-on garment, and the like, or any combinations thereof. Examples of pockets and spacers for use in absorbent products are described in U.S. Pat. No. 5,514,121; U.S. Pat. No. 5,171,236; U.S. Pat. No. 5,397,318; U.S. Pat. No. 5,540,671; U.S. Pat. No. 6,168,584; U.S. Pat. No. 5,306,266; and U.S. Pat. No. 5,997,520. Examples of compartments or voids in an absorbent article are disclosed in U.S. Pat. No. 4,968,312; U.S. Pat. No. 4,990,147; U.S. Pat. No. 5,062,840; and U.S. Pat. No. 5,269,755. Examples of suitable transverse barriers are described in U.S. Pat. No. 5,554,142; PCT Patent WO 94/14395; and U.S. Pat. No. 5,653,703. Examples of other structures suitable for management of low viscosity feces are disclosed in U.S. Pat. No. 5,941,864; U.S. Pat. No. 5,977,430; and U.S. Pat. No. 6,013,063.

While some of the individual features of some of the embodiments of the present invention have been known in the art, even greater benefits in urinary toilet training than those previously seen are possible using the combinations of elements described herein. Additionally, using some of the related features described can lead to even greater synergistic benefits. For example, refastenable fasteners with easy open tabs and the sensory elements described with active graphics can provide an article which a child may easily apply, check, remove (including when urination is imminent) and provides multiple sensory based feedback signals (such as visible graphics, tactile sensation, etc.). Such features re-enforce each other in ways previously not combined in the art.

As stated previously, the sensation member of the present invention can provide a wetness signal, a temperature change signal, or a combination thereof. For example, a portion of the liquid absorbed by the wetness sensation member can partially evaporate thereby causing a cooling sensation for the wearer. The evaporation of liquid can be enhanced where the backsheet or outer cover of the absorbent article includes a substantially vapor permeable material. For example, in some embodiments, the backsheet can be constructed to be permeable to at least water vapor and can have a moisture vapor transmission rate (MVTR) of at least 1000 g/m²/24 hr., preferably at least 1500 g/m²/24 hr., more preferably at least 2000 g/m²/24 hr., and even more preferably at least 3000 g/m²/24 hr. In some embodiments, the backsheet may define a moisture vapor transmission rate of from 1000 to 6000 g/m²/24 hr. or any individual number within the range. Some breathable backsheet materials are described in greater detail in PCT Application No. WO 95/16746; U.S. Pat. No. 5,938,648; U.S. Pat. No. 5,865,823; and U.S. Pat. No. 5,571,096. Other suitable exemplary materials and a suitable test method for measuring the MVTR is described in U.S. Pat. No. 6,448,467.

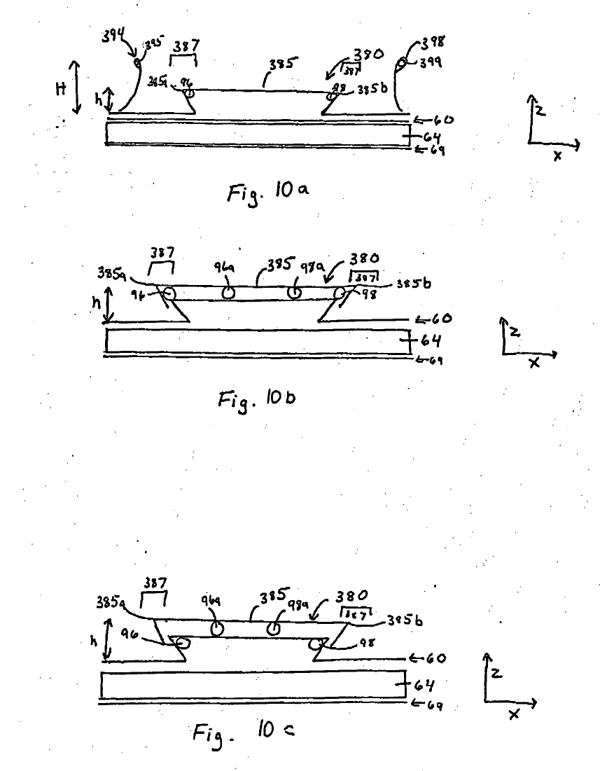

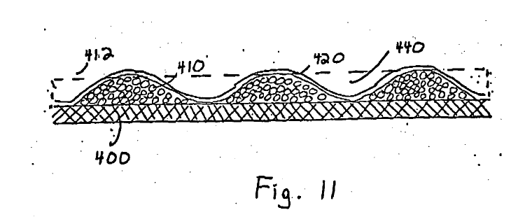

In additional embodiments shown in FIGS. 10A-10C, the sensation member 380 or any layer including the sensation member can be disposed in two parallel Z-folds 387 formed along the longitudinal length of the absorbent article. The Z-folded sensation member 380 or any layer including the sensation member may be attached to the underlying layers along the longitudinal edges of the topsheet 60 allowing the portion between the Z-folds of the topsheet 60 to float freely. Elastic elements 96, 98 may be disposed along the central region of the sensation member 380 in order to deflect the central region outward away from the absorbent core 64. Elastic elements 96, 98 may be disposed between layers of the topsheet 60, between layers of the sensation member 380, between the topsheet 60 and sensation member 380, or any other configuration that connects the elastic elements 96, 98 to the topsheet 60 and/or sensation member 380. The central region 385 may have a first side edge 385a and a second side edge 385b such that at least one of said side edges 385a, 385b has a projected height h measured the z direction between the side edge and the base of the sensation member that connects the sensation member to the absorbent article.

A disposable absorbent article including a sensation member is attached to the inner surface of a curved plate (i.e. the concave surface relative to the hypothetical center of the circle having the same curvature as the plate) having a radius of curvature of about 250 mm. The disposable absorbent article is attached to the plate such that its garment facing surface (i.e. outer cover) is in contact with the plate. In this configuration, the elastic member(s) that are disposed longitudinally on the disposable absorbent article are in an elongated configuration and are applying a force that is pulling any layer attached to the elastic member away from the core. A ruler having one end contacting the base of the sensation member and the other end pointing toward the center of the hypothetical circle formed by the curved plate, may be used to measure the distance between the base of the sensation and the side edge of the sensation member.

The Z-folded sensation member 380 allows the central region 385 to be suspended away from the core 64 and the topsheet 60. The combination of the Z-folded sensation member 380 and the elastic elements 96, 98 maintains the sensation members in proximity to the wearer's skin in the event that the diaper sags or fits loosely around the wearer.

Alternatively, additional elastic elements 96a, 98a may be disposed along the central region of the Z-folded sensation member. Elastic elements 96a, 98a, may be disposed between layers of topsheet 60, between layers of the sensation member 380, between the topsheet 60 and sensation member 380, or any other configuration that connects the elastic elements 96a, 98a to the topsheet 60 and/or sensation member 380. Elastic elements 96a, 98a provide additional support to prevent sagging and promote contact with the wearer's skin.

The absorbent article may also include a first barrier leg cuff 394 and a second barrier leg cuff 398, which may include elastic members 395, 399 respectively. First and second barrier leg cuffs are disposed on the absorbent article such that the Z-folded sensation member 380 is located between the barrier leg cuffs 395, 399. At least one of the first barrier leg cuff 394 and a second barrier leg cuff 398 has a projected height H measured the z direction between an upper edge of the barrier leg cuff and the base of the barrier leg cuff that connects the barrier leg cuff to the absorbent article.

The projected height h and H may be measured according the following method.

A disposable absorbent article including a sensation member is attached to the inner surface of a curved plate (i.e. the concave surface relative to the hypothetical center of the circle having the same curvature as the plate) having a radius of curvature of about 250 mm. The disposable absorbent article is attached to the plate such that its garment facing surface (i.e. outer cover) is in contact with the plate. In this configuration, the elastic member(s) that are disposed longitudinally on the disposable absorbent article are in an elongated configuration and are applying a force that is pulling any layer attached to the elastic member away from the core. A ruler having one end contacting the base of the sensation member and the other end pointing toward the center of the hypothetical circle formed by the curved plate, may be used to measure the distance between the base of the sensation and the side edge of the sensation member. The side edge of the sensation member is gently extended to its maximum height (i.e. without applying a force that would cause the sensation member to be torn or destroyed) and then record the measurement. The projected height measurement can be repeated at various points along the sensation member in order to determine its maximum projected height. The ruler may be moved such that one end is in contact with the base of an outer leg cuff and its other end is pointing towards the center of the hypothetical circle passing through the curved plate. The projected height H may be determined by measuring distance between the base of the outer leg cuff and the upper edge of the outer leg cuff. The upper edge of the outer leg cuff is gently extended to its maximum height (i.e. without applying a force that would cause the outer leg cuff to be tom or destroyed) and then record the measurement. The projected height measurement can be repeated at various points along the outer leg cuff in order to determine its maximum projected height.

In one embodiment, the projected height h of at least one of the first side edge 385a and a second side edge 385b is between 90% and 300%, preferably between 100% and 250%, more preferably between 100% and 200% of the projected height H of at least one of the first barrier leg cuff 394 and a second barrier leg cuff 398.

In one embodiment, the projected height h of at least one of the first side edge 385a and a second side edge 385b is between 15 mm and 50 mm, preferably between 20 mm and 45 mm, more preferably between 25 mm and 40 mm.

One preferred embodiment of the present invention includes, but is not limited to, articles described in U.S. Patent Application No. 2004/0162536 and U.S. Patent Application No. 2004/0167486. The aforementioned applications are directed to absorbent articles having an absorbent core which imparts increased wearing comfort to the article and makes it thin and dry. As shown in FIG. 11, the absorbent articles of the present invention may comprise an absorbent core 64 comprising a substrate layer 400, absorbent polymer material 410 and a fibrous layer of adhesive 420. The substrate layer 400 is preferably provided from a non-woven material, preferred non-wovens include those provided from synthetic fibers, such as PE, PET and PP. As the polymers used for non-woven production are inherently hydrophobic, they are preferably coated with hydrophilic coatings.

In accordance with the present invention, the absorbent material is immobilized when wet such that the absorbent core achieves a wet immobilization of more than 50%, preferably of more than 60%, 70%, 80% or 90%.

The substrate layer 400 comprises a first surface and a second surface. At least portions of the first surface of the substrate layer 400 are in direct contact with a layer of absorbent polymer material 410. This layer of absorbent polymer material 410 is preferably a discontinuous layer, and comprises a first surface and a second surface. As used herein, a discontinuous layer is a layer comprising openings. Typically, these openings have a diameter or largest span of less than 10 mm, preferably less than 5 mm, 3 mm, 2 mm and a span of more than 0.5 mm, 1 mm or 1.5 mm. At least portions of the second surface of the absorbent polymer material layer 410 are in contact with at least portions of the first surface of the substrate layer material 400. The first surface of the absorbent polymer material 410 defines a certain height 412 of the layer of absorbent polymer above the first surface of the layer of substrate material 400. When the absorbent polymer material layer 410 is provided as a discontinuous layer, portions of the first surface of the substrate layer 400 are not covered by absorbent polymer material 410. The absorbent core 64 further comprises a thermoplastic composition 420. This thermoplastic composition 420 serves to at least partially immobilize the absorbent polymer material 410.

In one preferred embodiment of the present invention the thermoplastic composition 420 can be disposed essentially uniformly within the polymeric absorbent material 410.

However, in an even more preferred embodiment of the present invention the thermoplastic material 420 is provided as a fibrous layer which is partially in contact with the absorbent polymer material 410 and partially in contact with the substrate layer 400. In this preferred structure the absorbent polymer material layer 410 is provided as a discontinuous layer, a layer of fibrous thermoplastic material 420 is laid down onto the layer of absorbent polymeric material 410, such that the thermoplastic layer 420 is in direct contact with the first surface of the layer of absorbent polymer material 410, but also in direct contact with the first surface of the substrate layer 400, where the substrate layer is not covered by the absorbent polymeric material 410. This imparts an essentially three-dimensional structure to the fibrous layer of thermoplastic material 420 which in itself is essentially a two-dimensional structure of relatively small thickness (in z-direction), as compared to the extension in x- and y-direction. In other words, the fibrous thermoplastic material layer 420 undulates between the first surface of the absorbent polymer material 410 and the first surface of the substrate layer 400.

Thereby, the thermoplastic material 420 provides cavities to hold the absorbent polymer material 410, and thereby immobilizes this material. In a further aspect, the thermoplastic material 420 bonds to the substrate 400 and thus affixes the absorbent polymer material 410 to the substrate 400. Highly preferred thermoplastic materials will also penetrate into both the absorbent polymer material 410 and the substrate layer 400, thus providing for further immobilization and affixation.

Of course, while the thermoplastic materials disclosed herein provide a much improved wet immobilization (i.e., immobilization of absorbent material when the article is wet or at least partially loaded), these thermoplastic materials also provide a very good immobilization of absorbent material when the article is dry.

In accordance with the present invention, the absorbent polymer material 410 may also be mixed with absorbent fibrous material, such as airfelt material, which can provide a matrix for further immobilization of the super-absorbent polymer material. However, preferably a relatively low amount of fibrous cellulose material is used, preferably less than 40 weight %, 20 weight %, or 10 weight % of cellulose fibrous material as compared to the weight of absorbent polymer material 410. Substantially airfelt free cores are preferred. As used herein, the term “absorbent fibrous material” is not meant to refer to any thermoplastic material 420 even if such thermoplastic material is fiberized and partially absorbent.

The absorbent core of the present invention may further comprise a cover layer. This cover layer may be provided of the same material as the substrate layer 400, or may be provided from a different material. Preferred materials for the cover layer are the non-woven materials. In this embodiment, portions of the cover layer bond to portions of the substrate layer 400 via the thermoplastic material 420. Thereby, the substrate layer 400 together with the cover layer provides cavities to immobilize the absorbent polymer material 410.

The areas of direct contact between the thermoplastic material 420 and the substrate material 400 are referred to as areas of junction 440. The shape, number, and disposition of the areas of junction 440 will influence the immobilization of the absorbent polymer material 410. The areas of junction can be of squared, rectangular, or circular shape. Preferred areas of junction are of circular shape. Preferably, they have a diameter of more than 0.5 mm, or 1 mm, or 1.5 mm and of less than 10 mm, or 5 mm, or 3 mm, or 2 mm. If the areas of junction 440 are not of circular shape, they preferably are of a size as to fit inside a circle of any of the preferred diameters given above.

The areas of junction 440 can be disposed in a regular or irregular pattern. For example, the areas of junction 440 may be disposed along lines. These lines may be aligned with the longitudinal axis of the absorbent core, or alternatively, they may have a certain angle in respect to the longitudinal edges of the core. It has been found, that a disposition along lines parallel with the longitudinal edges of the absorbent core 64 create channels in the longitudinal direction which lead to a lesser wet immobilization. Preferably, therefore the areas of junction 440 are arranged along lines which form an angle of 20 degree, 30 degree, 40 degree, or 45 degree with the longitudinal edges of the absorbent core 64. Another preferred pattern for the areas of junction 440 is a pattern comprising polygons, for example pentagons and hexagons or a combination of pentagons and hexagons. Also preferred are irregular patterns of areas of junction 440, which also have been found to give a good wet immobilization.

Two fundamentally different patterns of areas of junctions 440 can be chosen in accordance with the present invention. In one embodiment, the areas of junctions are discrete. They are positioned within the areas of absorbent material, like islands in a sea. The areas of absorbent materials are then referred to as connected areas. In an alternative embodiment, the areas of junctions can be connected. Then, the absorbent material can be deposited in a discrete pattern, or in other words the absorbent material represents islands in a sea of thermoplastic material 420. Hence, a discontinuous layer of absorbent polymer material 410 may comprise connected areas of absorbent polymer material 410 or may comprise discrete areas of absorbent polymer material 410.

In a further aspect of the present invention, it has been found that absorbent cores providing for a good wet immobilization can be formed by combining two layers. In this embodiment, the absorbent core material comprises two substrate layers 400, two layers of absorbent polymer material 410 and two layers of fibrous thermoplastic materials 420. When two discontinuous layers of an absorbent polymer material 410 are used, they would be typically arranged in such a way that the absorbent polymer material of the one layer faces the areas of junction 440 of the other layer. In an alternative preferred embodiment, however, the areas of junction 440 are offset and do not face each other.

According to the present invention, the thermoplastic layer 420 can comprise any thermoplastic composition, preferred are adhesive thermoplastic compositions, also referred to as hot melt adhesives. A variety of thermoplastic compositions are suitable to immobilize absorbent material. Some initially thermoplastic materials may later lose their thermoplasticity due to a curing step, e.g., initiated via heat, UV radiation, electron beam exposure or moisture or other means of curing, leading to the irreversible formation of a crosslinked network of covalent bonds. Those materials having lost their initial thermoplastic behaviour are herein also understood as thermoplastic materials 420.

In embodiments of the present disclosure, a disposable wearable absorbent article can include a stretchable outer cover. For example, the outer cover can be a uniaxially stretchable outer cover, configured to stretch in one direction. Also as an example, the outer cover can be a biaxially stretchable outer cover, configured to stretch in two directions. In various embodiments, the outer cover can be configured as described in US non-provisional patent application entitled “Biaxially Stretchable Outer Cover for an Absorbent Article,” filed on Nov. 15, 2006 with Express Mail No. EV916939625 and further identified by attorney docket number 10643, which is hereby incorporated by reference.

In embodiments of the present disclosure, a disposable wearable absorbent can include an outer cover configured in various ways, including configurations of part or all of the outer cover as stretchable, non-stretchable, with an elastic nonwoven, with an elastic film and extensible nonwoven, with an extensible film and an elastic nonwoven, pre-stretched with elastic strands allowed to contract, mechanically activated, with zero strain laminate, and/or combinations of these and any other outer cover configurations. In various embodiments of the present disclosure, a disposable wearable absorbent article can include a printed outer cover with various basis weights, chemistries, and/or mechanical activations, as will be understood by one of ordinary skill in the art.

### Measurement Methods

### Temperature Change Response Measurement

### Equipment

USB Data Acquisition System, OMB-DAQ-55 with Personal DAQView software from Omega Engineering Inc

| | |
|---|---|
| Thermocouples | −K type thermocouple, with a 6″ long, 0.020″ diameter, stainless steel |
| | sheath, ending in an exposed junction. (Part Number - KMTSS- |
| | 020E-6) from Omega Engineering Inc. |
| Computer | Suitable computer capable of running Personal DAQView Software. |
| Saline | 0.9% Saline heated to 37° C. +/− 1° C. |
| Syringe | Capacity of at least 75 mL |
| Timer | Convenient Source, time measurements taken to nearest second |
| Curved Plate | A U-shaped Plexiglas plate to mount the test product on in a curved |
| | configuration. A suitable plate is 5 mm thick, Radius of Curvature of |
| | the U-portion is 250 mm, Height (upright walls from bottom of “U”) |
| | is 160 mm, Width is 130 mm. The curved plate may be supported |
| | with side walls or other convenient mechanism to maintain upright, |
| | U-shaped disposition |

### Test Procedure

1. Test fluid is 0.9% saline heated to 37° C.±1° C.
2. If the product is provided in a closed, pant-like form, open side seams of all products. If product contains defined side seams product should be opened at those locations. Otherwise, cut side panels with scissors at midpoint of side panels.
3. Mount the test product onto the curved plate. Any suitable mechanism may be used. Two sided adhesive strips or hook material with the hooks facing the inside of the “U” may be used for this purpose depending upon the surface properties of the outer cover of the test product. (An example of hook material is supplied by Velcro USA Inc., Manchester, N.H. 03108). Typical hook or adhesive mounting strip is 520 mm long and 10 mm to 30 mm wide. Each strip is placed parallel to, and between 30 mm and 40 mm away from, with a strip on either side of the longitudinal center line of the template. The hook strips allow the continuous attachment of the test product to the template. The product is attached to the template such that the topsheet is facing away from the template (i.e. toward the space defined by the inside of the “U”). Pant elastics should stay intact.
4. The thermocouples are affixed on the surface using strips of masking tape 5 to 6 mm in width and 20 to 40 mm long. The closest tape is positioned such that the thermocouple tip is exposed and the longitudinal edge of the tape strip is between 4 and 6 mm from the thermocouple tip. This tape is then sued to attach the thermocouple on the surface to be measured such that the tip of the thermocouple is positioned no higher than 1 mm above said surface.
5. Measure as follows to define the loading point
   a. (boy) 10.2 cm below front edge of the core, or
   b. (girl) 12.7 cm below front edge of the core.
   c. (unisex or generic) use loading point measurement for boy products
6. Draw 75 mls of 0.9 saline (heated to 37° C.±1° C.) into syringe.
7. Temperature measurement is started between 5 to 15 seconds before the introduction of fluid and continues for at least the next 600 seconds.
8. Using syringe, deposit the saline at the loading point with a constant rate of loading. Loading should be done over approximately 5 seconds or at approximately 15 mL/second.
9. Once fluid is deposited, start timer. Pouring of the fluid is considered to be the “urination event” for comparison of time with time parameters in the claims.
10. Data is collected via the OMB-DAQ-55 system and stored in the computer in a predetermined folder, in various forms. One common for is an ASCII text file which is easily exported to common spreadsheet software (Excel) for further analysis.
11. Using generated data determine ΔT (taking 37° C. as Tₒ) at 30 seconds from start of timer.
12. Using generated data determine Tₘᵢₙ for 600 second interval. Determine ΔTₘₐₓ defined as 37° C.-Tₘᵢₙ. Duration of temperature change response is considered to be time at which thermocouple temperature is Tₘᵢₙ+25% ΔTₘₐₓ.

### Wetness Density Test

### Purpose

This test simulates the introduction of urine into a training pant diaper. No pressure is applied while loading to simulate the baby urinating in a standing position.

Equipment

| | |
|---|---|
| Template | Flat Base unit on which to mount the test product |
| Filter Paper | Ahlstrom Filtration Paper Code 632, 127 × 127 mm |
| Balance | accuracy +/−0.01 g |
| Saline | 0.9% Saline heated to 37° C. +/− 1° C. |
| Graduated | Convenient Source |
| Cylinder | |
| Timer | Convenient Source, time measurements taken to |
| | nearest second |
| Weight | Plastic Dimensions/Weight 127 × 127 mm, 297 grams |
| Metal Cylinder | Metal Cylinder, Inside Diameter = 60 mm, |
| | Outside Diameter = 70 mm, Height = 40 mm, |
| | Weight = 327 grams |

### Test Procedure

1. Test fluid is 0.9% saline heated to 37° C.±1° C.
2. If the product is provided in a closed, pant-like form, open side seams of all products. If product contains defined side seams product should be opened at those locations. Otherwise, cut side panels with scissors at midpoint of side panels.
3. Mount the test product with clamps onto a flat template in a flat stretched out condition to ensure wrinkles in the topsheet or liners. The pant elastics should stay intact.
4. Weigh one piece of filter paper.
5. Measure as follows to define the loading point (boy) 10.2 cm below front edge of the core, or (girl) 12.7 cm below front edge of the core. (unisex or generic) use loading point measurement for boy products
6. Measure 75 mls of 0.9 saline (heated to 37° C.±1° C.) into the graduated cylinder.
7. Center the cylinder over the loading point and pour the saline from the graduated cylinder at the loading point. Loading should be done over approximately 5 seconds or at approximately 15 mL/second.
8. Once fluid is poured, start timer. Pouring the saline is considered to be the “urination event” for purposes of comparison of time with time parameters in the claims.
9. After 60 seconds have elapsed, place filter paper on the topsheet and then the plastic weight to ensure complete contact between the filter paper and the topsheet. The weight should be lowered slowly and applied gently to the filter paper.
10. After 10 seconds from weight application, lift the weight and filter paper off of the topsheet and weigh the filter paper.
11. Calculate wet weight minus dry filter paper weight in mg. This value is divided by 161.29 cm² to determine the wetness density in mg/cm².

All documents cited in the Detailed Description are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

What is claimed is:

1. An absorbent article for wearing about the lower torso of a wearer said absorbent article comprising:
a first waist region, a second waist region disposed opposite said first waist region, a crotch region connecting said first waist region and said second waist region,
a water-impermeable outer cover, a water-permeable topsheet attached to said outer cover and having a body-facing surface;
an absorbent core disposed between said outer cover and said topsheet; and
a sensory element member at least partially disposed in said crotch region of said absorbent article, said sensory element member comprising a sensate providing a temperature sensation to said wearer upon a urination event, said sensory element member comprising a body-contacting portion.

2. The absorbent article as recited in claim 1, wherein said sensate comprises a coating.

3. The absorbent article as recited in claim 2, wherein said sensate is at least partially impregnated in said sensory element member.

4. The absorbent article as recited in claim 2, wherein said sensate is substantially impregnated in said sensory element member.

5. The absorbent article as recited in claim 1, wherein said sensate produces a sensation of a temperature decrease greater than an actual temperature decrease in response to exposure to urine.

6. The absorbent article as recited in claim 1, wherein said sensate is associated with said topsheet.

7. The absorbent article as recited in claim 6, wherein said sensate is coated onto said topsheet.

8. The absorbent article as recited in claim 6, wherein said sensate is at least partially impregnated into said topsheet.

9. The absorbent article as recited in claim 8, wherein said sensate is substantially impregnated into said topsheet.

10. The absorbent article as recited in claim 1, further comprising an auxiliary layer including said sensate, said auxiliary layer disposed proximal at least one of a body-facing surface and a garment-facing surface of said topsheet.

11. The absorbent article as recited in claim 10, wherein said sensate is at least partially impregnated into said auxiliary layer.

12. The absorbent article as recited in claim 10, wherein said sensate is substantially impregnated into said auxiliary layer.

13. The absorbent article as recited in claim 10, wherein said sensate is coated onto said auxiliary layer.

14. The absorbent article as recited in claim 1, wherein said sensate is selected from the group consisting of 1-(2-hydroxyphenyl-)-4-(2-nitrophenyl-)-1,2,3,6-tetrahydropyrimidine-2-one and derivatives thereof, ethyl 3-(p-menthane-3-carboxamido) acetate, N-(4-methoxyphenyl)-p-menthane-3-carboxamide, 1-glyceryl-p-menthane-3-carboxylate, ethylene glycol p-menthane-3-carboxylate, 3-(1-menthoxy)-2-methylpropane-1,2-diol, geraniol, eucalyptol, linaool, hydroxycitronellal, paramenthane-3,8-diol, menthyl pyrrolidone carboxlate, 2-isopropyl-5-methylcyclohexyl-4-(dimethylamino)-4-oxobutanoate, (−)-Cubebol; N-(4-cyanomenthylphenyl)-p-menthanecarboxamide, (1R,3R,4S)-3-menthyl-3,6-dioxaheptanoate, (1R,2S,5R)-3-menthyl-methoxyacetate, (1R,2S,5R)-3-menthyl-3,6,9-trioxadecanoate, (1R,2S,5R)-3-menthyl-(2-hydroxyethoxy)acetate, (1R,2S,5R)-menthyl-11-hydroxy-3,6,9-trioxaundacanoate, (−)-Isopulegol; cis and trans p-menthane-3,8-diol, and Cooling Agent 10 and derivatives, and combinations thereof.

15. The absorbent article as recited in claim 1, wherein said sensate comprises a hydrophilic material.

16. The absorbent article as recited in claim 1, wherein said sensate is a solid that dissolves in urine.

17. The absorbent article as recited in claim 1, wherein said sensate is selected form the group consisting of ketals, aliphatic carboxamides, cyclohexanol derivatives, and cyclohexyl derivatives.

18. The absorbent article as recited in claim 1, wherein said sensory element member further comprises at least one of a hydrophobic material, a hydrophilic material, and a tactile element.

19. The absorbent article as recited in claim 1, wherein said sensory element member further comprising a contact promoting substance.

20. The absorbent article as recited in claim 19, wherein said contact promoting substance is selected from the group consisting of a lotion, a sticky lotion, and an adhesive.

21. The absorbent article as recited in claim 1, wherein said article further comprises laterally extending stretchable ear panels.

22. The absorbent article as recited in claim 21, wherein said side panels are connected to form a closed waist opening.

23. The absorbent article as recited in claim 22, wherein said connection is refastenable and the article additionally comprises a fastener.

24. The absorbent article as recited in claim 22, wherein said side panels are connected via a hook component.

25. The absorbent article as recited in claim 1, wherein said ear panels comprising a pre-stretched elastic member.

26. The absorbent article as recited in claim 1, wherein said outer cover is breathable.

27. The absorbent article as recited in claim 1, further comprising at least one fastener configured to engage so as to define a waist opening, wherein said fastener is refastenable.

28. The absorbent article as recited in claim 1, further comprising standing leg cuffs extending outwardly therefrom.

29. The absorbent article as recited in claim 1, further comprising at least one of a temperature change material (or whatever our term is for agents that cause a real delta T), a wetness sensation element, and a tactile sensation element.

30. An absorbent article comprising:
an outer cover having a longitudinal axis;
a topsheet attached to the outer cover and having a body-facing surface and an opposite surface facing the outer cover;
an absorbent core disposed between the outer cover and the topsheet;
a sensation member comprising a sensate being spaced a distance upwardly from said absorbent core.

31. The absorbent article as recited in claim 30, further comprising associated elastic elements that space said sensate from said core.

32. The absorbent article as recited in claim 30, wherein said sensate is applied to a region of the topsheet on one of the body-facing surface and the opposite surface, the region of the topsheet

33. The absorbent article as recited in claim 30, wherein said sensate is coated onto said body-facing surface of said topsheet.

34. The absorbent article as recited in claim 30, wherein said sensate is at least partially impregnated into said topsheet.

35. The absorbent article as recited in claim 30, wherein said sensate is disposed in one or more zones, said zones being spaced from each other with respect to at least one of an x-direction, a y-direction, and a z-direction.

36. The absorbent article as recited in claim 30, wherein said sensation member further comprises at least one of a hydrophilic material, a hydrophobic material, and a tactile material.

37. The absorbent article as recited in claim 30, further comprising first and second spaced barrier leg cuffs attached to the topsheet parallel to the longitudinal axis, said topsheet being to said barrier leg cuffs such that said body-facing surface of said topsheet is spaced upwardly from said absorbent core.

38. An insert removably attachable to an absorbent article, the insert comprising:
a first structural layer, said first structural layer being liquid permeable;
a sensate associated with at least one of a body-facing surface and a garment-facing surface of said structural layer; and
a fastening member configured to removably connect to the absorbent article.

39. The insert as recited in claim 38, further comprising a core disposed at the garment-facing surface of said structural layer.

40. The insert as recited in claim 38, further comprising a second structural layer disposed beneath the core such that the core is disposed between the first and second structural layers.

41. The insert as recited in claim 40, further comprising an elastic member disposed between the core and the first structural layer.

42. The insert as recited in claim 38, wherein said sensate is coated onto said first structural layer.

43. The insert as recited in claim 38, wherein said sensate is at least partially impregnated into said first structural layer.

44. The insert as recited in claim 43, wherein said sensate is substantially impregnated into said first structural layer.
